(19) United States
(12) Patent Application Publication
(10) Pub. No.: US 2015/0217518 A1
(43) Pub. Date: Aug. 6, 2015

(54) Construction Of A Facial Mask For Air Supply And Air Exchange

(71) Applicant: Hankookin, Inc., Raleigh, NC (US)

(72) Inventors: Andrew Youngho Chun, Raleigh, NC (US); James Jiwen Chun, Raleigh, NC (US); Angela Soyoung Chun, Raleigh, NC (US); Jennifer Miseong Chun, Raleigh, NC (US)

(73) Assignee: Hankookin, Inc.

(21) Appl. No.: 14/612,348

(22) Filed: Feb. 3, 2015

Related U.S. Application Data
(60) Provisional application No. 61/934,859, filed on Feb. 3, 2014.

(51) Int. Cl. B29C 67/00; G06F 17/50; G05B 15/02; A61M 16/06

(52) U.S. Cl. CPC B29C 67/0088; A61M 16/0616; G06F 17/50; G05B 15/02; A61M 2207/10; B33Y 50/02

(57) ABSTRACT

A computer implemented method and a mask development system (MDS) for constructing a three-dimensional (3D) facial mask for air supply and air exchange are provided. The MDS receives images of a face and biometric data, constructs a 3D facial structure of the face's actual size using one or more facial parameters obtained from the received images and biometric data, and constructs a 3D mask structure that fits on internal and/or external areas of one or more facial parts of the 3D facial structure. The MDS configures the 3D mask structure by creating a seal around the internal and/or external areas, configuring grooves proximal to the internal and/or external areas for incorporating air supply and air exchange elements, and configuring design parameters, in the 3D mask structure. The MDS transmits the configured 3D mask structure to a 3D printer for constructing the 3D facial mask for air supply and air exchange.

### CROSS REFERENCE TO RELATED APPLICATIONS

This application claims priority to and the benefit of provisional patent application No. 61/934,859 titled “Three-dimensional Facial Mask With Provisions For Air Supply And Exchange”, filed in the United States Patent and Trademark Office on Feb. 3, 2014. The specification of the above referenced patent application is incorporated herein by reference in its entirety.

### BACKGROUND

With the advent of three-dimensional (3D) scanning, 3D designing, and 3D printing technologies, functionality and aesthetic appeal of conventional personal equipment can be maximized. For example, personal and medical equipment such as medical masks have been used to provide clean air to a user through the user's nostrils. However, typical medical masks that function by providing air through the nostrils fit poorly around the facial area and provide limited space for an exchange of a sufficient and a comfortable quantity of buffering air in front of the nostril area. Moreover, a high performance surgical mask, for example, a surgical N95 respirator or a N99 particulate filtering facepiece respirator certified by the National Institute for Occupational Safety and Health (NIOSH) is hard to breathe through because air suction pressure or air discharge pressure created by human lungs is not strong enough for breathing in air or receiving sufficient air flow through air filters. Furthermore, these surgical masks do not provide adequate space for facial movements. Furthermore, the appearance of such surgical masks is not aesthetic.

Even though aesthetics play a major role in human nature, design of conventional facial masks that provide an aesthetic appeal has been overlooked. Typically, conventional facial masks are not designed to be minimally visible and therefore are easily noticeable when worn by users in public. Therefore, when users wear conventional facial masks in public, the facial masks are conspicuously embarrassing. Medical equipment such as continuous positive airway pressure (CPAP) masks that provide a regulated supply of oxygen at a high level to patients who suffer from oxygen depletion during sleep cycles, are typically uncomfortable to wear while sleeping. Equipment such as the surgical N95 respirators, the N99 particulate filtering facepiece respirators, and the CPAP masks have a similar function of delivering air to the lungs, but provide an ill fit, an unpleasant appearance, and discomfort during use. Absence of an adequate seal between an ill-fitting mask and the facial area can allow inward leakage of ambient air, contaminants, etc. Moreover, defects comprising, for example, facial scars, missing teeth, a broken nose, etc., in facial features can prevent a proper fit of the mask.

Patients using continuous positive airway pressure (CPAP) masks typically experience nasal congestion, nasal dryness, throat dryness, etc., due to air being blown into the nasal area throughout the night. Furthermore, some patients feel restricted while wearing the CPAP masks due to insufficient spaces between the CPAP masks and the nasal areas. Conventionally, patients suffering from oxygen depletion during sleep cycles prefer to use oral appliances to ease breathing during sleep. Oral appliances are typically available in different designs and help in keeping the airway open during sleep. However, these oral appliances fail to provide a custom fit on the intraoral areas comprising, for example, teeth, alveolar ridges, etc. Moreover, these oral appliances fail to filter air breathed by a patient via the patient′ mouth.

Hence, there is a long felt but unresolved need for a computer implemented method and system that constructs a three-dimensional (3D) facial mask for air supply and air exchange, that fits comfortably on one or more facial parts of a user's face, and that is functional, aesthetically pleasing, and can be implemented for personal and medical usage. Moreover, there is a need for a computer implemented method and system that constructs a 3D facial mask that can be customized to fit well on one or more facial parts of the user's face, with air supply and air exchange elements to facilitate ease of breathing and facial movements. Furthermore, there is a need for a computer implemented method and system that constructs a 3D facial mask for air supply and air exchange, that provides a comfort fit for the intranasal areas and/or the intraoral areas of a user's face, is minimally visible, and filters air breathed by the user.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The computer implemented method and system disclosed herein addresses the above stated need for constructing a three-dimensional (3D) facial mask for air supply and air exchange, that fits comfortably on one or more facial parts of a user's face, and that is functional, aesthetically pleasing, and can be implemented for personal and medical usage. Moreover, the computer implemented method and system disclosed herein constructs a 3D facial mask that can be customized to fit well on one or more facial parts of a user's face, with air supply and air exchange elements to facilitate ease of breathing and facial movements. Furthermore, the computer implemented method and system disclosed herein constructs a 3D facial mask for air supply and air exchange, that provides a comfort fit for the intranasal areas and/or the intraoral areas of a user's face, is minimally visible, and filters air breathed by the user.

The computer implemented method disclosed herein employs a computer implemented mask development system, hereinafter referred to as a “mask development system”, comprising at least one processor configured to execute computer program instructions for constructing a three-dimensional (3D) facial mask for air supply and air exchange. The mask development system receives multiple images of a user's face and biometric data comprising, for example, the user's height and the user's facial dimensions. The mask development system constructs a 3D facial structure of an actual size of the user's face using one or more of multiple facial parameters obtained from the received images and the biometric data. The mask development system constructs a 3D mask structure configured to fit internal areas and/or external areas of one or more facial parts of the constructed 3D facial structure. The facial parts of the constructed 3D facial structure correspond, for example, to a nose and a mouth of the user's face. The internal areas comprise, for example, intranasal areas and intraoral areas. The external areas comprise, for example, extranasal areas and extraoral areas.

The mask development system configures the constructed three-dimensional (3D) mask structure by creating a seal in the constructed 3D mask structure, around the internal areas and/or the external areas of one or more facial parts of the constructed 3D facial structure. The mask development system further configures the constructed 3D mask structure by configuring one or more grooves in the constructed 3D mask structure, proximal to the internal areas and/or the external areas of one or more facial parts of the constructed 3D facial structure for incorporating one or more of multiple air supply and air exchange elements, for example, an in-tube, an out tube, a safety plug, a filter element, etc. The mask development system further configures the constructed 3D mask structure by configuring one or more design parameters, for example, facial characteristics, physical dimensions, color, size, shape, one or more design patterns, one or more accessories, etc., for the constructed 3D mask structure. In an embodiment, the mask development system further configures the constructed 3D mask structure by configuring one or more spaces in the constructed 3D mask structure, around the internal areas and/or the external areas of one or more facial parts of the constructed 3D facial structure, using one or more of the facial parameters obtained from the received images and the biometric data for enabling ease of breathing and facial movements. The mask development system transmits the configured 3D mask structure to a 3D printing device for constructing the 3D facial mask for air supply and air exchange.

In one or more embodiments, related systems include but are not limited to circuitry and/or programming for effecting the methods referenced herein; the circuitry and/or programming can be any combination of hardware, software, and/or firmware configured to effect the herein-referenced methods depending upon the design choices of a system designer. Also, various structural elements may be employed depending on the design choices of the system designer.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1A illustrates a computer implemented method for constructing a three-dimensional facial mask for air supply and air exchange.

FIG. 1B illustrates an embodiment of the computer implemented method for constructing a three-dimensional facial mask for air supply and air exchange.

FIG. 2 exemplarily illustrates a left side perspective view of a three-dimensional mask structure configured to fit one or more external areas of one or more facial parts of a three-dimensional facial structure constructed using a mask development system.

FIG. 3 exemplarily illustrates a perspective view of a three-dimensional facial mask constructed using the mask development system, showing incorporation of air supply and air exchange elements in the three-dimensional facial mask.

FIGS. 4A-4B exemplarily illustrate front elevation views of personalized three-dimensional facial masks with enhanced aesthetic appeal, constructed using the mask development system.

FIGS. 5A-5B exemplarily illustrate front elevation views of three-dimensional facial masks configured with multiple design patterns using the mask development system.

FIG. 5C exemplarily illustrate a side perspective view of a three-dimensional facial mask configured with a design pattern and advertisements using the mask development system for public communication.

FIGS. 6A-6B exemplarily illustrate different views of a filter element for filtering air passing through intraoral areas of a mouth of a face of a user.

FIG. 7 exemplarily illustrates a side perspective view of a mouth of a face of a user, showing incorporation of the filter element inside the mouth for filtering air passing through intraoral areas of the mouth of the user.

FIGS. 8A-8B exemplarily illustrate side perspective views of a three-dimensional facial mask constructed using the mask development system for intraoral areas of a mouth of a face of a user.

FIG. 9 exemplarily illustrates a side perspective view of a three-dimensional facial mask constructed using the mask development system for external areas of a nose of a face of a user, in conjunction with the three-dimensional facial mask constructed for intraoral areas of a mouth of the face.

FIG. 10 exemplarily illustrates a perspective view of a three-dimensional facial mask constructed using the mask development system for intranasal areas of a nose of a face of a user.

FIG. 11 exemplarily illustrates an exploded view of the three-dimensional facial mask constructed using the mask development system for intranasal areas of a nose of a face of a user.

FIGS. 12A-12B exemplarily illustrate different views of the three-dimensional facial mask shown in FIG. 10, inserted into the intranasal areas of a nose of a face of a user for filtering air passing through the intranasal areas.

FIG. 13 exemplarily illustrates a three-dimensional facial mask constructed using the mask development system for external areas of a mouth of a face of a user, in conjunction with the three-dimensional facial mask constructed for intranasal areas of a nose of the face.

FIG. 14 exemplarily illustrates a front perspective view of an embodiment of the three-dimensional facial mask shown in FIG. 10, constructed using the mask development system for intranasal areas of a nose of a face of a user.

FIGS. 15A-15B exemplarily illustrate different views of the three-dimensional facial mask shown in FIG. 14, inserted into the intranasal areas of a nose of a face of a user for filtering air passing through the intranasal areas.

FIG. 16 exemplarily illustrates a computer implemented system for constructing a three-dimensional facial mask for air supply and air exchange.

FIG. 17 exemplarily illustrates the hardware architecture of the mask development system employed for constructing a three-dimensional facial mask for air supply and air exchange.

### DETAILED DESCRIPTION OF THE INVENTION

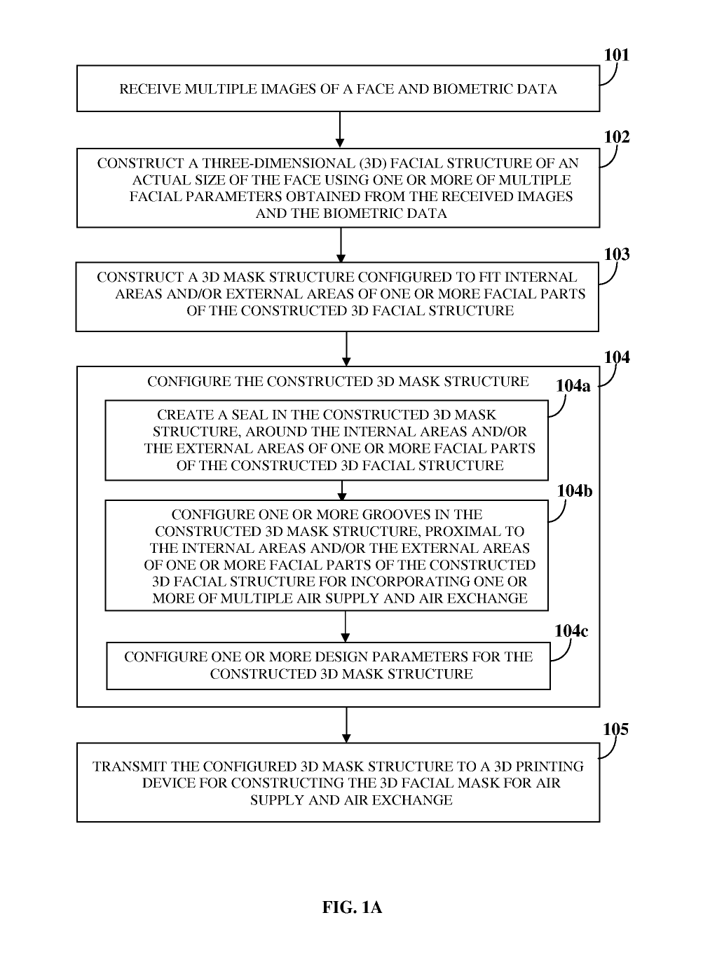

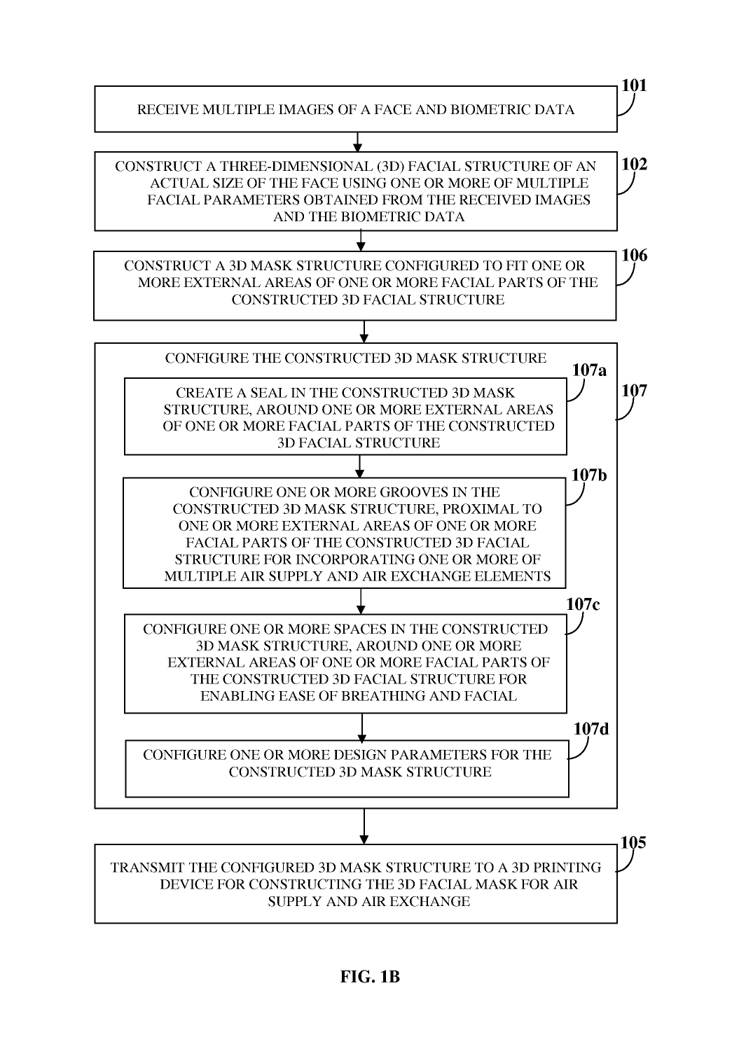

FIG. 1A illustrates a computer implemented method for constructing a three-dimensional (3D) facial mask for air supply and air exchange. As used herein, “3D facial mask” refers to a structure configured to cover and provide a sealed comfort fit around one or more areas, for example, intranasal areas, intraoral areas, extranasal areas, extraoral areas, etc., of one or more facial parts, for example, a nose, a mouth, etc., of a user's face for protecting the user from inhaling ambient air, air pollutants, contaminants, infection, chemicals, etc., by filtering air that the user breathes through this structure, or for enabling the user to conceal and/or enhance one or more facial features of the user. Also, as used herein, the term “user” refers to a person who wears the 3D facial mask for medical purposes, decorative purposes, cultural purposes, advertising purposes, etc. The “user” may also refer to a mask developer, a mask designer, a mask customizer, etc., who provides user inputs to construct and develop the 3D facial mask. The computer implemented method disclosed herein employs a computer implemented mask development system, hereinafter referred to as a “mask development system”, comprising at least one processor configured to execute computer program instructions for constructing the 3D facial mask for air supply and air exchange. The mask development system constructs 3D designed facial masks that have a seal, for example, around nasal areas such as nostrils, allow facial movements such as lip movements and chin movements for maximal function as a 3D facial mask, and provide maximal comfort, while allowing optimal spaces for air supply and air exchange and easing facial movements. As used herein, “air supply and air exchange” refer to supply of pure air into the 3D facial mask, flow of pure or inhaled air into the 3D facial mask, and flow of impure or exhaled air out of the 3D facial mask. The mask development system uses 3D technology to construct a customized 3D facial mask that has an aesthetic appearance and provides a comfort fit to a user such as a patient, a medical practitioner, an advertiser, etc., who wears the 3D facial mask. The mask development system constructs a 3D facial mask of different types that can be used alone or in combination with other 3D facial masks for covering and filtering internal areas and/or external areas of one or more facial parts of the user's face.

The mask development system receives 101 multiple images of a user's face and biometric data comprising, for example, a user's height and a user's facial dimensions from the user or a user device, for example, a mobile phone, a smart phone, a tablet computing device, an image capture device such as a camera, one or more biometric devices, etc. As used herein, “biometric data” refers to data used to identify a user based on the user's physical traits or behavioral characteristics. The biometric data comprises, for example, facial patterns, facial dimensions, height measurements, etc. The images of the user's face comprise multiple views of the user's face. The views comprise, for example, an elevation view such as a full face view, a profile view, etc. The mask development system receives multiple two-dimensional (2D) images and/or three-dimensional (3D) images of the user's face by employing 2D and/or 3D image capture devices, for example, 2D and/or 3D cameras. Development of the fitting 3D facial mask starts with the mask development system obtaining 2D and/or 3D images of the user's face. For obtaining the 3D images of the user, the mask development system employs many methods and technologies, for example, using front view and side view photographs of the user, a full size image of the user, the user's biometric data, etc. The mask development system can also use photometric methods to obtain a preliminary 3D image of the user.

The mask development system constructs 102 a three-dimensional (3D) facial structure of an actual size of the user's face using one or more of multiple facial parameters obtained from the received images and the biometric data. As used herein, “facial parameters” refer to angular and ratiometric measurements of the user's facial features. The facial parameters comprise, for example, a nasal tip projection, a cheekbone projection, an angle between the user's forehead and the user's nose, a distance between the nose and the user's mouth, a chin projection, a nose ridge form, a width of the nose, a length of the nose, dimensions of nostrils of the nose, a distance between the nostrils, a chin shape, a lip profile, a dental profile comprising dimensions of teeth and an alveolar ridge form, etc. Using the received images, for example, a full size image of the user and the biometric data such as the user's height and the user's facial dimensions for size calibration, the mask development system obtains an actual sized 3D facial image or structure of the user. The biometric data comprising, for example, facial dimensions such as a facial width, a facial height, and a nose width helps the mask development system to improve the accuracy of the actual sized 3D facial image or structure of the user. The mask development system uses the user's height for size calibration of the full size image of the user. From an accurate measurement of the full size image of the user, the mask development system obtains an estimate of the size of the user's face by measuring the facial dimensions in the full size image of the user.

The mask development system measures the facial dimensions from the received images by applying one or more mathematical principles comprising, for example, trigonometry principles and geometry principles to the received images and the biometric data. The mask development system implements multiple algorithms that define methods for constructing a 3D facial structure, for example, from 2D images in different views based on photogrammetry, for example, by using Photomodeler® of Eos Systems Inc., a software application that performs image based modeling and close range photogrammetry for producing 3D models and facial measurements from 2D photography of the user's face.

Consider an example where a user provides a full size image of the user's body to the mask development system and wants to construct a three-dimensional (3D) facial structure of an actual size of the user's face. The mask development system receives the full size body image of the user and the biometric data comprising, for example, a measurement of the user's body height from the user or the user device. Consider an example where the actual height of the user's body is 170 cm, while the height of the user's body in the received image is 8 cm. The mask development system maps the dimensions of the user's body height in the received image, that is, 8 cm to the received biometric data, that is, 170 cm. For example, the mask development system scales the facial dimensions in the received image based on a ratio of the received biometric data, that is, the actual height of the user's body to the dimensions of the user's body height in the received image. In this example, the ratio is calculated as 170/8=21.25. Therefore, if the facial length in the received image is 0.8 cm, then the mask development system constructs a 3D facial structure with a facial length=0.8*21.25=17 cm.

The mask development system constructs 103 a three-dimensional (3D) mask structure configured to fit internal areas and/or external areas of one or more facial parts of the constructed 3D facial structure. As used herein, “facial parts” refer to different parts of a user's face that define multiple features of the face. The facial parts comprise, for example, a nose, a mouth, a chin, a jaw, cheeks, etc. The facial parts of the constructed 3D facial structure correspond, for example, to a nose and a mouth of a user's face. Also, as used herein, “internal areas” refer to concealed or partially visible areas positioned inside the facial parts. The internal areas comprise, for example, intranasal areas such as a nasal septum, nasal cavities extending from the nostrils of a user's nose, etc., intraoral areas such as an oral cavity, an oral palate, a tongue, teeth, gingiva, alveolar ridges, etc. The internal areas are typically concealed by the facial parts and therefore have low visibility as compared to the external areas of the facial parts. Also, as used herein, “external areas” refer to visible areas of the facial parts that are covered with dermis, that is, skin. The external areas comprise, for example, extranasal areas such as extranasal dermal tissue, philtrum, etc., extraoral areas such as lips, and other external areas such as the user's chin, cheeks, etc.

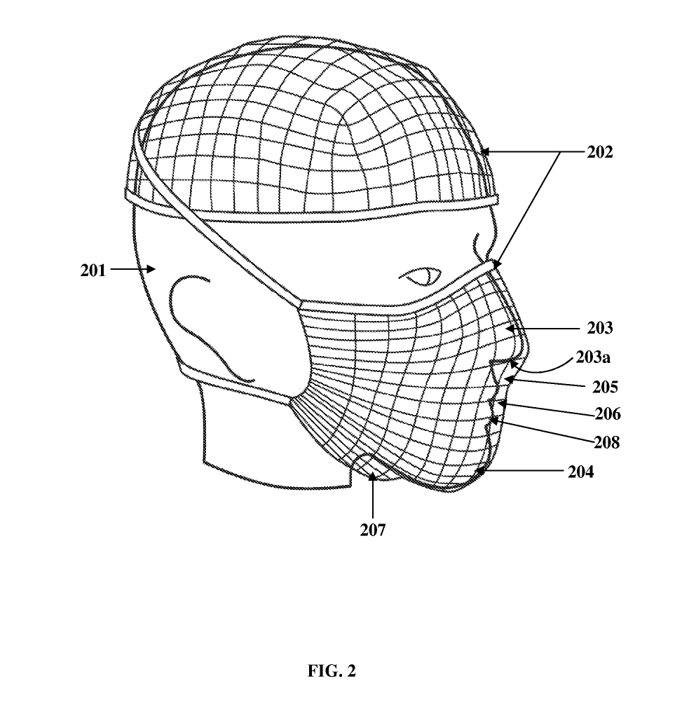

For construction of the three-dimensional (3D) mask structure, the mask development system uses the constructed 3D facial structure as a reference. The 3D facial structure is, for example, a model of an actual size of the user's head with the user's face as disclosed in the detailed description of FIG. 2. Once the 3D facial structure is constructed, the mask development system can construct multiple 3D mask structures configured to fit internal areas and/or external areas of one or more facial parts of the constructed 3D facial structure. For example, if a user wants to develop a respiratory 3D facial mask that covers the nose and the mouth of the user's face, then the mask development system constructs a 3D mask structure that fits on the external areas of the user's nose and the user's mouth, for example, by using an external face profile of the user's nose and the user's mouth in the constructed 3D facial structure. In another example, if the user wants to develop a respiratory 3D facial mask covering only the nose of the user's face, then the mask development system constructs a 3D mask structure that fits on the external areas of the nose of the constructed 3D facial structure. In another example, if the user wants to develop a 3D facial mask covering only the mouth of the user's face, then the mask development system constructs a 3D mask structure that fits on the external areas of the mouth of the constructed 3D facial structure. In another example, if the user wants to develop a 3D facial mask for filtering air flow through the user's nose, then the mask development system constructs a 3D mask structure that fits in the intranasal areas such as the nostrils of the nose of the constructed 3D facial structure. In another example, if the user wants to develop a 3D facial mask for filtering air flow through the user's teeth, then the mask development system constructs a 3D mask structure comprising a teeth tray that fits on an intraoral area, for example, the teeth inside the mouth of the constructed 3D facial structure. In another example, if the user wants to develop a 3D facial mask for filtering air flow through the user's intraoral areas, then the mask development system constructs a 3D mask structure comprising a teeth tray and an intraoral filter paper that fits inside the mouth of the constructed 3D facial structure. Thus, using a single 3D facial structure, the mask development system constructs multiple 3D mask structures configured to fit the internal areas and/or the external areas of one or more facial parts of the 3D facial structure, thereby optimizing the time required in development of 3D facial masks of multiple sizes and shapes that cover multiple different facial parts.

The mask development system configures 104 the constructed 3D mask structure. For configuring the constructed 3D mask structure, the mask development system creates 104a a seal in the constructed 3D mask structure, around the internal areas and/or the external areas of one or more facial parts of the constructed 3D facial structure. As the constructed 3D mask structure is configured to fit the internal areas and/or the external areas of the facial parts of the constructed 3D facial structure, the constructed 3D mask structure comprises an exact replica of a section of the constructed 3D facial structure. This section comprises, for example, external areas of a nose and a mouth that are to be covered by the 3D facial mask. Therefore, this section of the constructed 3D mask structure comprises parts corresponding exactly to the facial parts of constructed 3D facial structure. Thus, the mask development system uses the internal areas and/or the external areas of the facial parts of the constructed 3D facial structure as references to determine the corresponding internal areas and/or external areas on the constructed 3D mask structure to configure the constructed 3D mask structure, for example, to create a seal around the determined internal areas and/or the determined external areas in the constructed 3D mask structure.

In an embodiment, the mask development system creates a seal around the nasal areas, for example, the external areas of the nose of the constructed 3D facial structure, in the constructed 3D mask structure. In another embodiment, the mask development system creates a seal around a periphery of the constructed 3D mask structure. In this embodiment, the periphery comprises, for example, the nasal ridge, cheek areas, and chin areas of the constructed 3D facial structure. The mask development system further configures 104 the constructed 3D facial structure by configuring 104b one or more grooves in the constructed 3D mask structure, proximal to the internal areas and/or the external areas of one or more facial parts of the constructed 3D facial structure for incorporating air supply and air exchange elements. The air supply and air exchange elements comprise, for example, an in-tube, an out tube, a safety plug, a filter element, etc. As used herein, “filter element” refers to an element configured to filter air passing through the element. The filter element is, for example, an intraoral filter paper, mesh elements, etc.

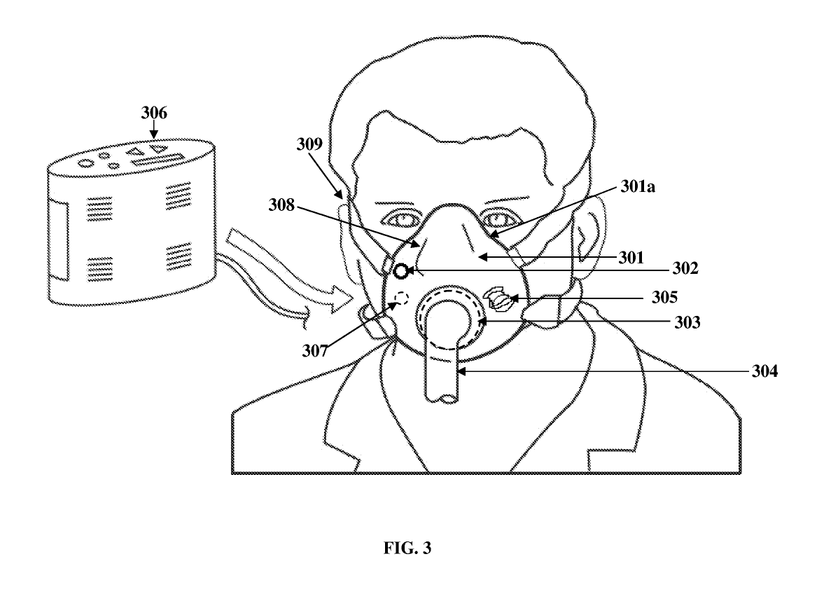

In an embodiment, the mask development system positions the grooves, for example, above and below the nasal area of the constructed 3D facial structure for constructing a 3D facial mask that covers the external areas of the nose or the external areas of the nose and the mouth. In this embodiment, the grooves configured by the mask development system in the constructed 3D mask structure allow clean air or oxygen rich air to enter through an in-tube. In order to balance air pressure inside the 3D facial mask to be constructed, the mask development system makes provisions in the constructed 3D mask structure to let out exhaled air through an out tube. Since the exhaled air goes forward from the nasal area, that is, nostrils, and fresh air comes from all around the nasal area, the mask development system positions a groove for the in-tube above the nasal area and a groove for the out tube below the nasal area for air supply and air exchange as disclosed in the detailed description of FIG. 3.

In an embodiment, the mask development system further configures the constructed 3D mask structure by configuring one or more spaces in the constructed 3D mask structure, around the internal areas and/or the external areas of one or more facial parts of the constructed 3D facial structure using one or more of the facial parameters obtained from the received images and the biometric data of the user for enabling ease of breathing and facial movements. The mask development system configures these spaces by using the constructed 3D facial structure as a reference and by using the obtained facial parameters to determine the dimensions of the spaces to be configured between the constructed 3D facial structure and the constructed 3D mask structure. For example, the mask development system creates an open space in front of the external areas such as nostrils of the nose of the constructed 3D facial structure by design and additional spaces around muscle movement areas, for example, around the lip and the chin of the constructed 3D facial structure as disclosed in the detailed description of FIG. 2. In an embodiment, the mask development system creates spaces around the internal areas, for example, the teeth for enhancing dental features and/or accommodating dental prostheses such as clear braces that are not visible. For example, for a user with an uneven set of teeth, the mask development system configures the constructed 3D mask structure with one or more spaces around one or more teeth in order to enhance an aesthetic appeal of the dental profile of the user.

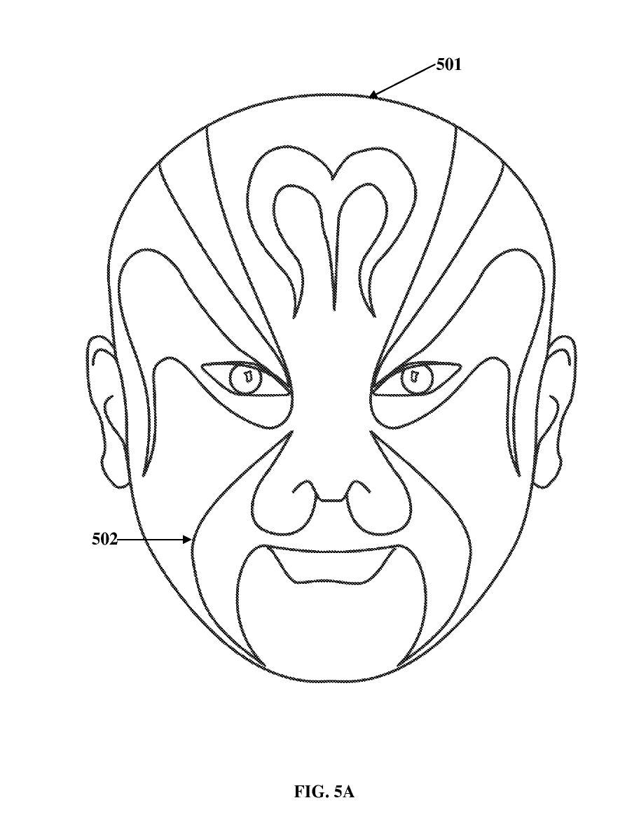

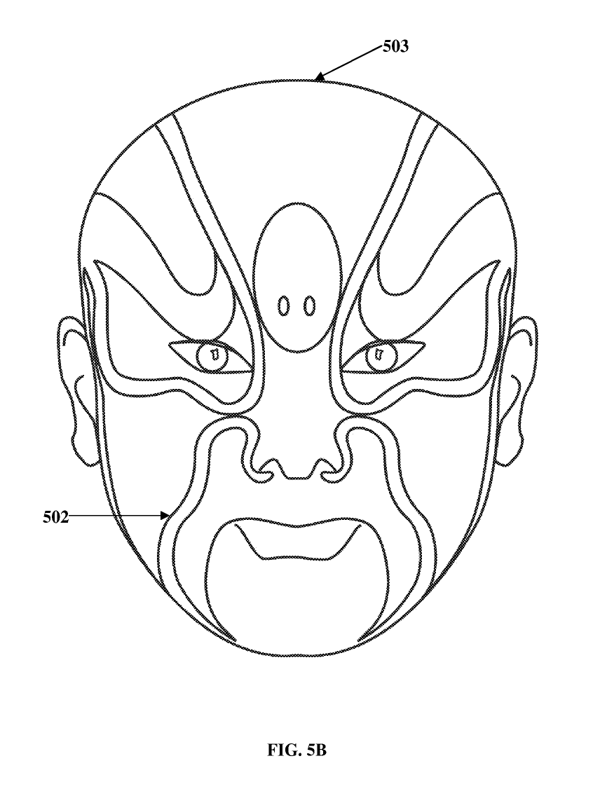

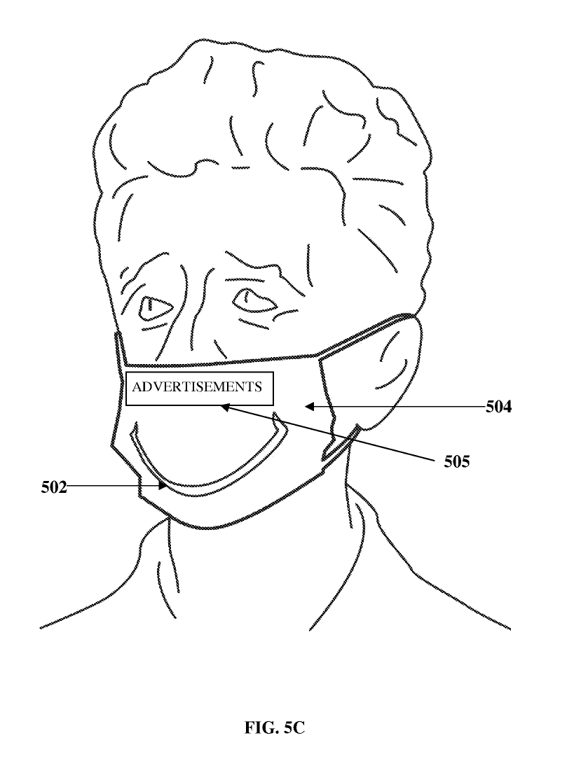

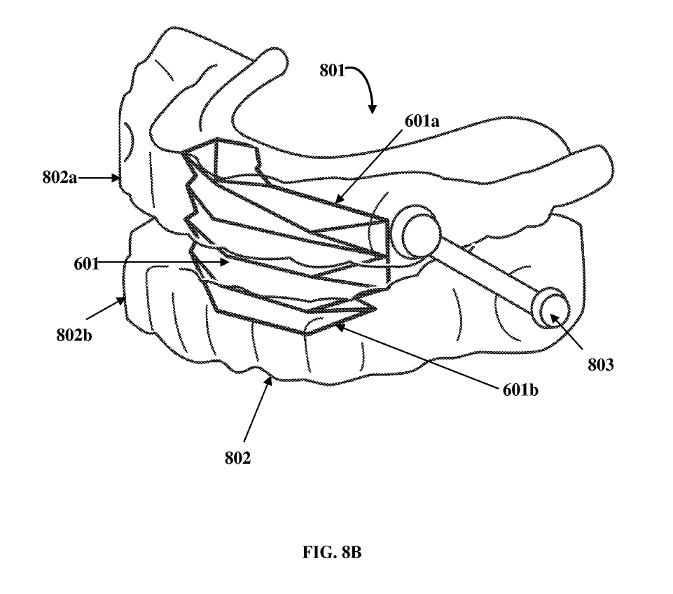

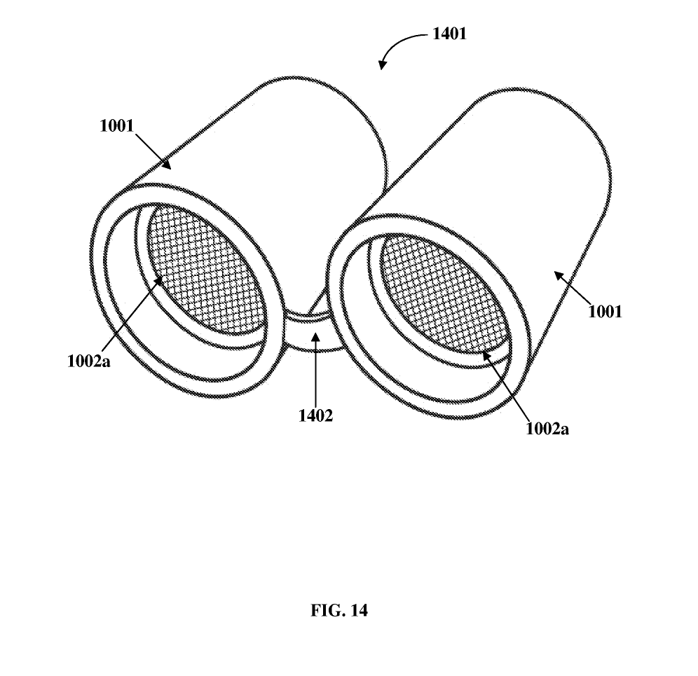

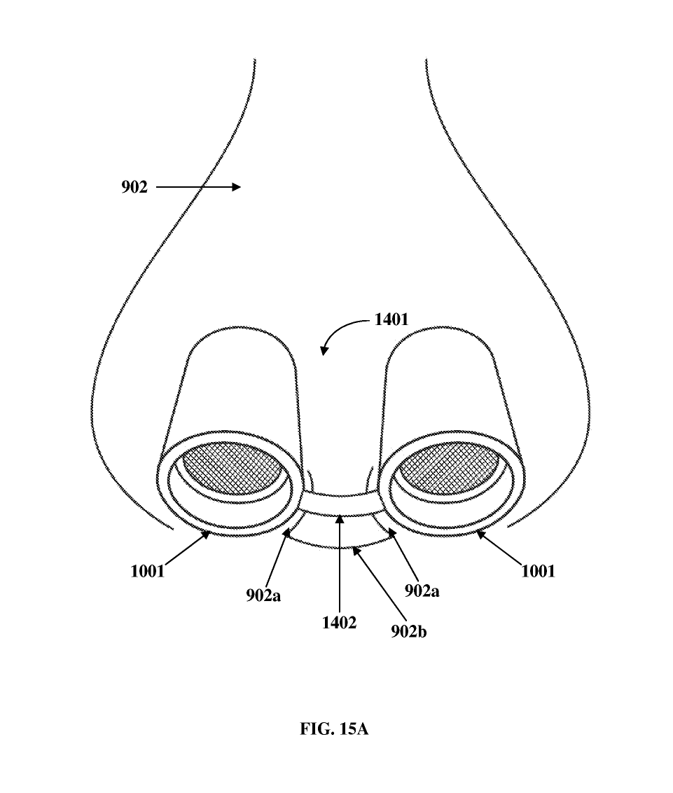

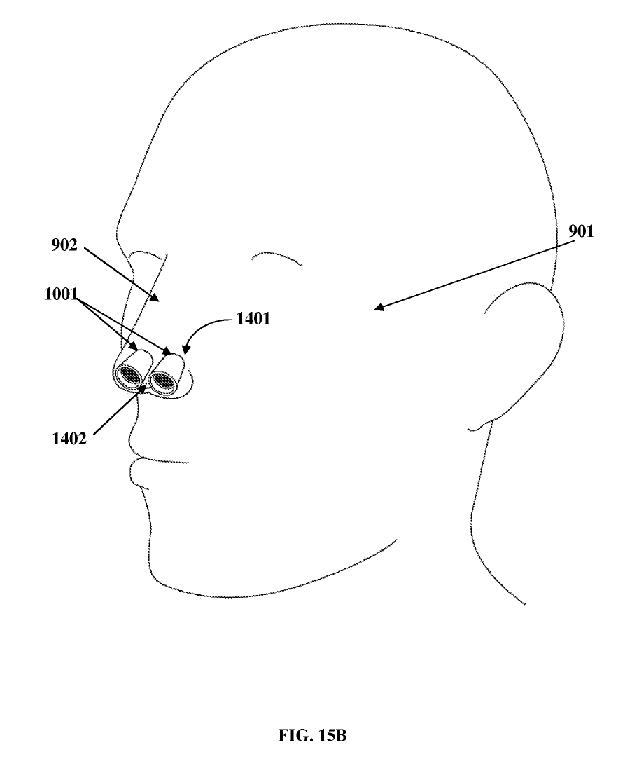

The mask development system further configures 104 the constructed 3D mask structure by configuring 104c one or more design parameters for the constructed 3D mask structure. As used herein, “design parameters” refer to design aspects, for example, an aesthetic appearance, an area of the 3D constructed mask structure such as a nasal area that the 3D facial mask is intended to cover, etc., used for designing a 3D facial mask to enhance the aesthetic appeal of the 3D facial mask. The mask development system configures the design parameters based on user information comprising gender of the user, age of the user, etc., one or more user inputs, etc., received from the user or the user device. The design parameters comprise, for example, facial characteristics, physical dimensions, color, size, shape, one or more design patterns, one or more accessories, etc. As used herein, “facial characteristics” refer to one or more parts of a face that can be personalized based on a user's preference while configuring the constructed 3D mask structure. The facial characteristics comprise, for example, a nose ridge form, a chin shape, a lip profile, etc. Also, as used herein, “physical dimensions” refer to dimensions of areas that are sealed and covered by the 3D facial mask. The physical dimensions comprise, for example, size of cheek bones, a circumference of an oral area, a periphery of a nasal area, etc. The facial characteristics and physical dimensions vary according to gender, for example, male or female, of the user wearing the 3D facial mask. Also, as used herein, the term “accessories” refers to supplementary elements configured to be positioned on the 3D facial mask for enhancing functionality and/or the aesthetic appeal of the 3D facial mask. The accessories are, for example, a decorative item, a strap, a clip attached to the 3D facial mask for facilitating an adjustable comfort fit of the 3D facial mask on the user's face as disclosed in the detailed description of FIG. 14 and FIGS. 15A-15B, a lever for adjusting the position of the 3D facial mask as disclosed in the detailed description of FIGS. 8A-8B, etc. The mask development system configures the constructed 3D mask structure for incorporating one or more accessories. Configuring design parameters for the 3D facial mask increases the aesthetic appeal of the 3D facial mask as disclosed in the detailed description of FIGS. 5A-5B.

In an embodiment, the mask development system configures the constructed three-dimensional (3D) mask structure in one or more image formats comprising, for example, an object (obj) file format, stereolithography (STL) file format, etc., which can be converted into image formats that can be printed using 3D printing devices, for example, 3D printers. The mask development system transmits 105 the configured 3D mask structure to a 3D printing device, for example, a 3D printer such as MakerBot® of MakerBot Industries, LLC, Cube® of 3D Systems, Inc., etc., for constructing the 3D facial mask for air supply and air exchange. The mask development system can construct a 3D facial mask for covering only a nasal area, only an oral area, or both a nasal area and an oral area of a user's face. The mask development system can further construct a 3D facial mask for covering other internal areas and/or external areas of the user's face. A user wearing the 3D facial mask can breathe either through the nose, the mouth, or both the nose and the mouth without any discomfort.

FIG. 1B illustrates an embodiment of the computer implemented method for constructing a three-dimensional (3D) facial mask for air supply and air exchange. In this embodiment, the computer implemented method disclosed herein constructs a 3D facial mask for external areas comprising, for example, extranasal areas and/or extraoral areas of one or more facial parts comprising, for example, a nose and/or a mouth of a user's face. The mask development system receives 101 multiple images of the user's face and the user's biometric data comprising, for example, the user's height, the user's facial dimensions, etc. The mask development system constructs 102 a 3D facial structure of an actual size of the user's face using one or more of multiple facial parameters, for example, a nasal tip projection, a cheekbone projection, an angle between a forehead and a nose, a distance between the nose and a mouth, a chin projection, a nose ridge form, a chin shape, and a lip profile, obtained from the received images and the biometric data. The mask development system constructs 106 a 3D mask structure configured to fit one or more external areas of one or more facial parts of the constructed 3D facial structure. For example, the mask development system constructs a 3D mask structure configured to fit extranasal areas and/or extraoral areas of the nose and/or the mouth of the constructed 3D facial structure.

The mask development system configures 107 the constructed 3D mask structure. For configuring the constructed 3D mask structure, the mask development system creates 107a a seal in the constructed 3D mask structure, around the external areas of one or more facial parts of the constructed 3D facial structure. For example, the mask development system creates a seal in the constructed 3D mask structure, around the extranasal areas of the nose of the constructed 3D facial structure. The mask development system further configures 107 the constructed 3D mask structure by configuring 107b one or more grooves in the constructed 3D mask structure, proximal to the external areas of one or more facial parts of the constructed 3D facial structure for incorporating one or more air supply and air exchange elements. For example, the mask development system configures a groove in the constructed 3D mask structure above the extranasal area of the constructed 3D facial structure for incorporating an in-tube and configures another groove in the constructed 3D mask structure below the extranasal area of the constructed 3D facial structure for incorporating an out tube as disclosed in the detailed description of FIG. 3. The mask development system further configures 107 the constructed 3D mask structure by configuring 107c one or more spaces in the constructed 3D mask structure, around the external areas of one or more facial parts of the constructed 3D facial structure using the facial parameters obtained from the received images and the biometric data for enabling ease of breathing and facial movements. For example, the mask development system configures spaces in front of the extranasal areas such as nostrils of the nose of the constructed 3D facial structure and additional spaces around the lip and the chin of the constructed 3D facial structure as disclosed in the detailed description of FIG. 2. The mask development system further configures 107 the constructed 3D mask structure by configuring 107d one or more design parameters, for example, a design pattern for the constructed 3D mask structure. The mask development system transmits 105 the configured 3D mask structure to a 3D printing device for constructing the 3D facial mask for air supply and air exchange.

FIG. 2 exemplarily illustrates a left side perspective view of a three-dimensional (3D) mask structure 202 configured to fit one or more external areas of one or more facial parts, for example, a nose 203 and a chin 204 of a 3D facial structure 201 constructed using the mask development system. The mask development system configures spaces 205 around the extranasal areas of the nose 203, spaces 206 around extraoral areas, for example, oral muscle areas, of a mouth 208, and spaces 207 around chin areas of the chin 204, etc., in the 3D mask structure 202 for enabling ease of breathing and facial movements. For example, the mask development system configures extra spaces 205 and 206 in front of the nostrils 203a and the mouth 208, in the 3D mask structure 202 for ease of lip movements, and an extra space 207 around the chin 204 for ease of facial movements comprising, for example, jaw movements, chin movements, neck movements, etc.

Consider an example where a user wants a three-dimensional (3D) facial mask of an actual size of his/her face for air supply and air exchange, where the 3D facial mask covers a nose and a mouth of the user's face. The user provides multiple two-dimensional (2D) images in multiple views comprising, for example, a profile view, an elevation view, etc., of the user's face to the mask development system. The mask development system receives the 2D images of the user's face from the user and obtains one or more facial parameters, for example, a nasal tip projection, an angle between a forehead and the nose, a distance between the nose and a mouth, a chin projection, a nose ridge form, a chin shape, a lip profile, etc., from the received 2D images. The mask development system constructs a 3D facial structure 201 of an actual size of the user's face using the obtained facial parameters. The mask development system constructs a 3D mask structure 202 configured to fit one or more extranasal areas and extraoral areas of the nose 203 and the mouth 208 of the constructed 3D facial structure 201. The mask development system configures the constructed 3D mask structure 202 by creating a seal around the extranasal areas and/or the extraoral areas and configuring one or more grooves proximal to the extranasal areas and/or the extraoral areas in the constructed 3D mask structure 202. In this example, if the user wants to incorporate an in-tube (not shown) above the extranasal area and an out tube 304 exemplarily illustrated in FIG. 3, below the extranasal area of the constructed 3D facial structure 201 for facilitating air supply and air exchange in the 3D facial mask, the mask development system configures a groove 302 above the extranasal area in the constructed 3D mask structure 202 for incorporating the in-tube that supplies fresh air for inhalation, and a groove 303 below the extranasal area in the constructed 3D mask structure 202 for incorporating the out tube 304 that exhausts air that is exhaled. The mask development system further configures the constructed 3D mask structure 202 by configuring spaces 205, 206, and 207 around the extranasal areas of the nose 203, extraoral areas of the mouth 208, and the chin areas of the chin 204 of the constructed 3D facial structure 201 respectively, using the obtained facial parameters for enabling ease of breathing and facial movements. The user provides his/her inputs for one or more design parameters comprising, for example, a red color, a plastic material, a floral pattern, etc., based on the user's preferences to the mask development system to configure the design parameters for the constructed 3D mask structure 202. The mask development system configures the design parameters for the constructed 3D mask structure 202 based on the user inputs and transmits the configured 3D mask structure 202 to a 3D printing device for constructing the 3D facial mask for air supply and air exchange.

FIG. 3 exemplarily illustrates a perspective view of a three-dimensional (3D) facial mask 301 constructed using the mask development system, showing incorporation of air supply and air exchange elements, for example, an out tube 304 and a safety plug 305 in the 3D facial mask 301. The 3D facial mask 301 comprises a seal 308 created around the extranasal areas of the user's nose. The mask development system configures one or more grooves 302 and 303 proximal to the extranasal areas of the nose 203 and the extraoral areas of the mouth 208 of the constructed 3D facial structure 201 exemplarily illustrated in FIG. 2, while configuring the constructed 3D mask structure 202 exemplarily illustrated in FIG. 2, to incorporate the air supply and air exchange elements. As exemplarily illustrated in FIG. 3, the mask development system configures a groove 302 for incorporating an in-tube (not shown), a groove 303 for incorporating an out tube 304, and a groove (not shown) for incorporating the safety plug 305. The user may introduce the in-tube and the out tube 304 into the 3D facial mask 301 for air supply and air exchange, through an ear line 309 of the user's face similar to positioning of temples of a frame of a pair of eye glasses for aesthetic appeal. Since the 3D facial mask 301 constructed using the mask development system is sealed, oxygen air pressure inside the 3D facial mask 301 is maintained at a designated level to avoid oxygen depletion. For example, the oxygen concentration inside the 3D facial mask 301 is maintained at a designated oxygen air pressure level of about 21% by volume of air, to avoid discomfort to the user wearing the 3D facial mask 301. An optimal amount of oxygen flow required to maintain the oxygen concentration at the designated oxygen air pressure level inside the 3D facial mask 301 can be obtained by performing tests, for example, a test to determine an average rate of inhalation and exhalation on different users of the 3D facial mask 301.

For air supply and air exchange, the three-dimensional (3D) facial mask 301 constructed using the mask development system allows introduction of filtered air via air supply filters 306 similar to, for example, air filtered through fabrics used in N95 masks, N99 masks, etc., or highly oxygenated air supplied by an oxygen tank or an oxygen concentrator. The mask development system allows incorporation of air supply and air exchange elements, for example, an in-tube (not shown) to feed air into the 3D facial mask 301 from the air supply filters 306 as a continuous flow or a pulsed flow. Moisture is maintained in the 3D facial mask 301 at a comfortable level, for example, moisture at a level preset between about 20% and about 70% of the air or other gas supplied in the 3D facial mask 301. The mask development system makes provisions to keep the level of moisture, that is, relative humidity inside the 3D facial mask 301 adjustable since a comfort level for relative humidity varies substantially from user to user. The mask development system allows incorporation of one or more sensors, for example, a humidity sensor (not shown) and a regulator (not shown) inside the 3D facial mask 301 to facilitate adjustment of relative humidity inside the 3D facial mask 301.

An air supply filter 306, for example, a portable air filter or an oxygen generator that can be carried around by the user, can be used to supply air through the in-tube inserted in the groove 302 of the 3D facial mask 301. The air supply filter 306 uses a battery to pump air through a N95 filter or a N99 filter to clean the air. The air supplied by the air supply filter 306 to the 3D facial mask 301 via the in-tube is either in a continuous flow or a pulsed flow. The air supply filter 306 may also use zeolite to absorb nitrogen gas at a high atmospheric pressure, for example, of about 2 atmospheres to about 3 atmospheres and provide high oxygen concentrated air continuously to the user wearing the 3D facial mask 301 via the in-tube. The out tube 304 is connected to an air suction pump (not shown) that suctions out air from the 3D facial mask 301 via the out tube 304 to balance the air pressure inside the 3D facial mask 301.

The safety plug 305 is configured to open the seal 308 in the 3D facial mask 301 when the level of oxygen air pressure inside the 3D facial mask 301 drops below the designated oxygen air pressure level by being unplugged manually or automatically. In an embodiment, the mask development system makes provisions in the 3D facial mask 301 to incorporate or accommodate an air pressure sensor 307. In this embodiment, the air pressure sensor 307 continuously monitors the oxygen air pressure inside the 3D facial mask 301 and generates an alert to notify a user wearing the 3D facial mask 301 to unplug the safety plug 305, when the oxygen air pressure inside the 3D facial mask 301 drops below an optimum threshold level, that is, the designated oxygen air pressure level. In another embodiment, the air pressure sensor 307 automatically unplugs the safety plug 305 to open the seal 308 in the 3D facial mask 301. The material used to manufacture the 3D facial mask 301 is, for example, either similar to the material used in filters for masks such as the N95 mask, the N99 mask, etc., or plastic to enable ease of breathing and facial movements. Unlike the masks used for providing a continuous positive airway pressure (CPAP), the mask development system provides a seal 308 around the 3D facial mask 301. This seal 308 applies a light pressure to hold the 3D facial mask 301 on a user's face without creating any distortion around facial tissues under an edge 301a of the 3D facial mask 301.

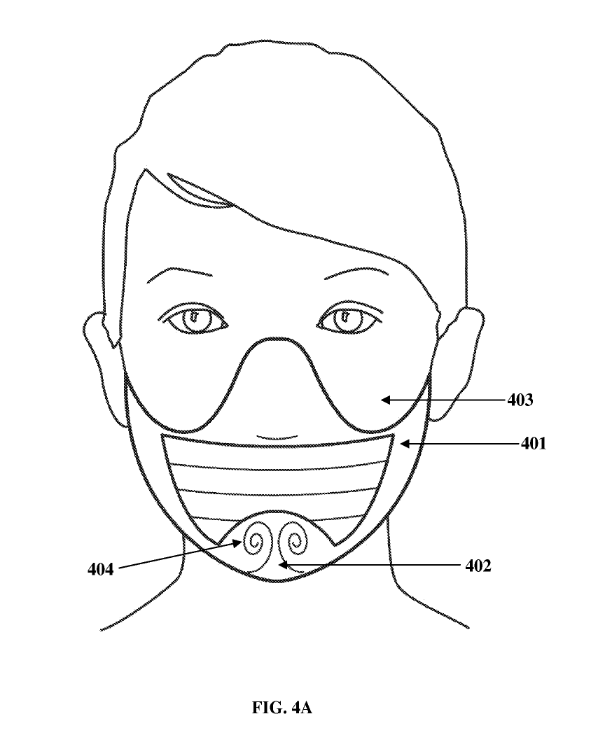

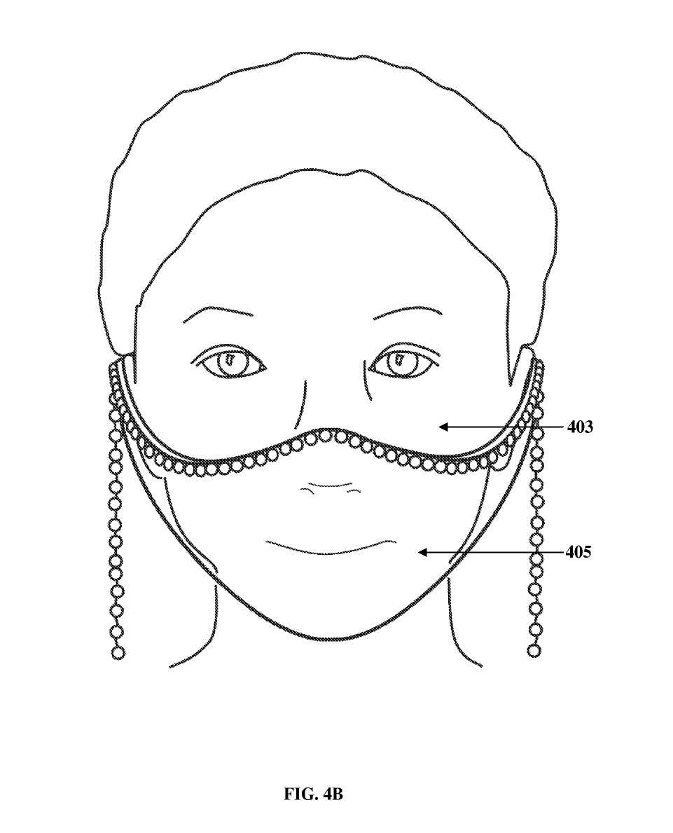

FIGS. 4A-4B exemplarily illustrate front elevation views of personalized three-dimensional (3D) facial masks 401 and 405 with enhanced aesthetic appeal, constructed using the mask development system. Aesthetic considerations for the 3D facial masks 401 and 405 are based on functional structures configured in the 3D facial masks 401 and 405. The mask development system configures design parameters, for example, a design pattern 404 for the 3D facial mask 401 exemplarily illustrated in FIG. 4A, to provide maximal and diversified aesthetic appeal to the 3D facial mask 401. The functional structures in the 3D facial mask 401 comprise sections for accommodating a user's nose, the user's mouth, and air supply and air exchange elements comprising, for example, an out tube 304 and a safety plug 305 exemplarily illustrated in FIG. 3.

The mask development system configures face profiles for constructing 3D facial masks 401 and 405 that fit onto existing facial structures of users without disrupting the users' overall facial profiles, in which relative proportions of the existing facial structures are maintained. The mask development system constructs a face profile, for example, by constructing a 3D facial structure 201 exemplarily illustrated in FIG. 2, in which relative proportions of the user's facial parameters are maintained for reference to construct the 3D facial masks 401 and 405. Deficiencies or disproportions in a user's existing facial structure can be modified while configuring the constructed 3D mask structure 202 on the constructed 3D facial structure 201 exemplarily illustrated in FIG. 2, by redesigning the constructed 3D mask structure 202. For example, for a user with a deficient lower chin, the mask development system configures the constructed 3D mask structure 202 with a bigger or an elongate chin area on the constructed 3D facial structure 201 for constructing a 3D facial mask 401 with a bigger or an elongate chin area 402 and an enhanced aesthetic appeal as exemplarily illustrated in FIG. 4A. The mask development system uses, for example, colors, shades, design pattern lines, and geometrical forms to create an artistic appeal for the 3D facial masks 401 and 405. The mask development system incorporates facial profiles that most users find aesthetically appealing, while configuring the constructed 3D mask structures 202 on the constructed 3D facial structure 201. The mask development system incorporates these facial profiles during construction of the 3D facial structure 201 and the 3D mask structure 202. The mask development system also creates an overall shape and form for the 3D mask structure 202 in an artistic manner as exemplarily illustrated in FIG. 4A.

The mask development system characterizes and personalizes different facial parts to create aesthetically appealing 3D facial masks 401 and 405. The mask development system redesigns facial characteristics such as the nose ridge form, the chin shape, and the lip profile to increase the aesthetic appeal of the 3D facial mask 401 as exemplarily illustrated in FIG. 4A. The facial structures of women and men are different. The facial muscle areas 403 defining cheek bones of female users, exemplarily illustrated in FIGS. 4A-4B, express more emotions than the facial muscle areas (not shown) defining cheek bones of male users. Thus, the mask development system exposes optimal areas 403 defining the cheek bones in the 3D facial mask 401 depending on whether the 3D facial mask 401 is worn by a male user or a female user. For male users with a strong jaw line and an angular face, the mask development system constructs an armor shaped 3D facial mask 401 exposing the jaw line and chin areas for a strong appeal as exemplarily illustrated in FIGS. 5A-5B.

FIGS. 5A-5B exemplarily illustrate front elevation views of three-dimensional (3D) facial masks 501 and 503 configured with multiple design patterns 502 using the mask development system. The mask development system configures design patterns 502 comprising, for example, folklore art, cultural arts symbols, other art design patterns, etc., on the 3D facial masks 501 and 503 for public communication.

FIG. 5C exemplarily illustrate a side perspective view of a three-dimensional (3D) facial mask 504 configured with a design pattern 502 and advertisements 505 using the mask development system for public communication. The mask development system receives the design pattern 502, images of one or more advertisements 505, and one or more locations for positioning the design pattern 502 and the advertisements 505 on the constructed 3D facial mask 504 from the user or the user device. The mask development system positions the design pattern 502 and the images of the advertisements 505 at the locations provided by the user on the constructed 3D mask structure 202 exemplarily illustrated in FIG. 2, while configuring the constructed 3D mask structure 202 on the constructed 3D facial structure 201 exemplarily illustrated in FIG. 2. The mask development system then transmits the configured 3D mask structure 202 with the design pattern 502 and the advertisements 505 to a 3D printing device, for example, a 3D printer such as MakerBot® of MakerBot Industries, LLC, for constructing the 3D facial mask 504.

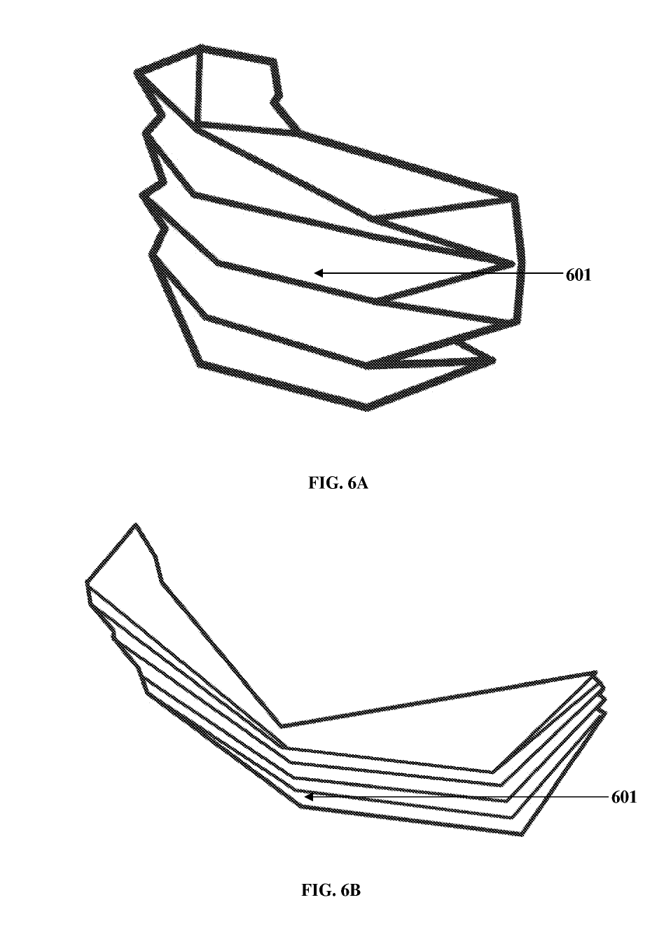

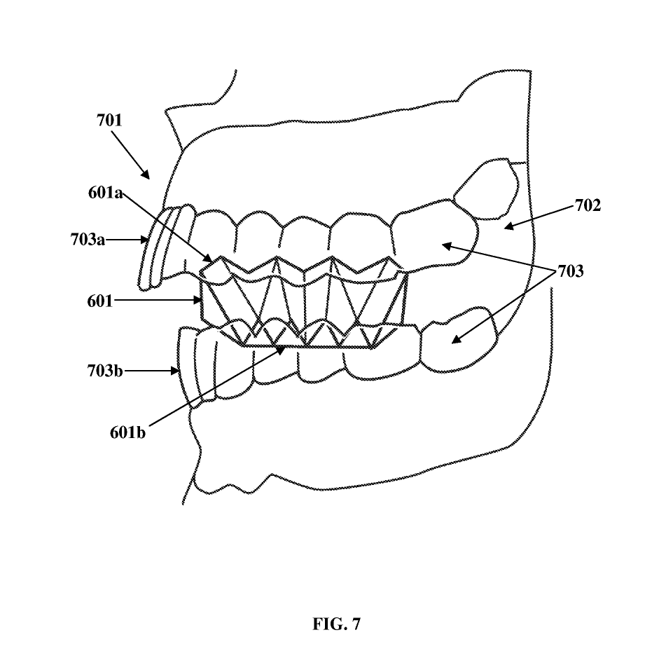

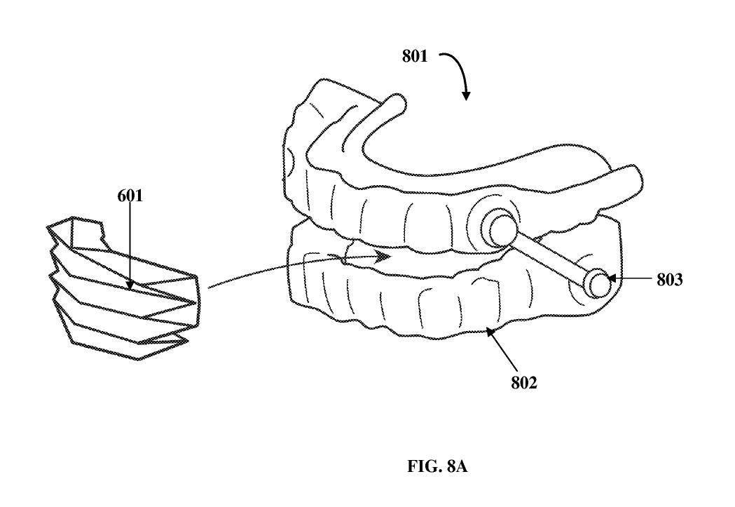

FIGS. 6A-6B exemplarily illustrate different views of a filter element, for example, an intraoral filter paper 601 for filtering air passing through intraoral areas 701 of a mouth 701 of a user's face exemplarily illustrated in FIG. 7. In one or more embodiments, the intraoral filter paper 601 is configured in multiple sizes and made of one or more materials comprising, for example, fabric, metal, plastic, etc. The intraoral filter paper 601 is folded into multiple small folds that can be stretched as exemplarily illustrated in FIG. 6A, and recoiled as exemplarily illustrated in FIG. 6B, when a user's mouth 701 is opened and closed respectively.

FIG. 7 exemplarily illustrates a side perspective view of a mouth 701 of a user's face, showing incorporation of the filter element, for example, the intraoral filter paper 601 inside the user's mouth 701 for filtering air passing through intraoral areas 702 comprising, for example, the oral cavity of the user's mouth 701. Since a tongue needs maximum space to function, an optimal position for the intraoral filter paper 601 is behind the teeth 703 in the user's mouth 701. The intraoral filter paper 601 is stacked behind the user's teeth 703 such that the opposing edges 601a and 601b of the intraoral filter paper 601 are in contact with the upper teeth 703a and the lower teeth 703b respectively as exemplarily illustrated in FIG. 7. Since the mouth opening, that is, the intraoral areas 702, is larger than the openings in the intranasal areas such as nostrils, air flows easily through the intraoral filter paper 601. The saliva inside the intraoral areas 702 moistens air inside the intraoral areas 702 and in turn moistens the intraoral filter paper 601. The moistening of the intraoral filter paper 601 enables the intraoral filter paper 601 to adhere to the teeth 703 and remain in a fixed position.

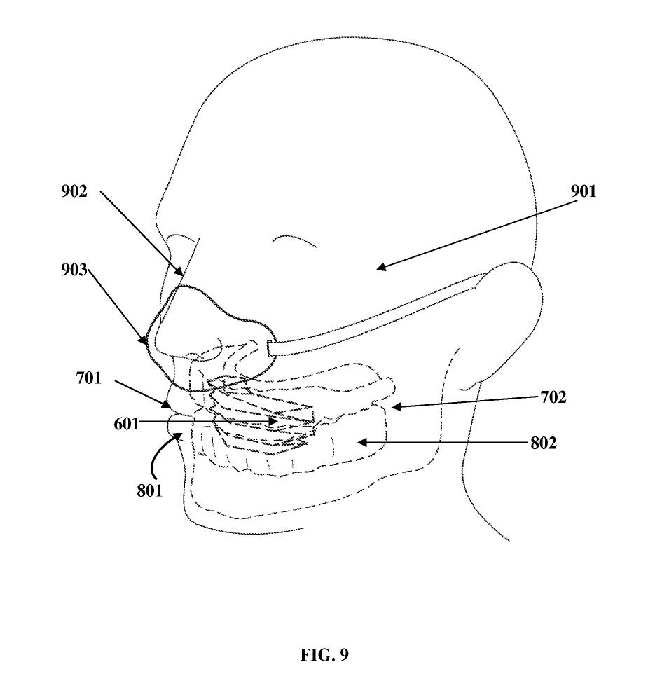

FIGS. 8A-8B exemplarily illustrate side perspective views of a three-dimensional (3D) facial mask 801 constructed using the mask development system for intraoral areas 702 of a mouth 701 of a user's face 901 exemplarily illustrated in FIG. 9. The 3D facial mask 801 comprises a filter element, for example, the intraoral filter paper 601 and an intraoral teeth tray 802 for precluding leakage of air between the teeth 703 exemplarily illustrated in FIG. 7, while filtering air passing through the intraoral areas 702 of the mouth 701. The 3D facial mask 801 is an intraoral 3D facial mask constructed for covering one or more internal areas, for example, intraoral areas 702 of the user's mouth 701. The teeth tray 802 is configured to accommodate teeth 703 inside the intraoral areas 702. The teeth tray 802 prevents air from leaking between gaps (not shown) between the user's teeth 703. The teeth tray 802 is positioned to fit snugly over the teeth 703 and is used in conjunction with the intraoral filter paper 601 for directing air flow to the intraoral filter paper 601 to ensure filtered air flow through the intraoral areas 702. The intraoral filter paper 601 is inserted between the upper portion 802a of the teeth tray 802 and the lower portion 802b of the teeth tray 802 as exemplarily illustrated in FIG. 8B, such that the opposing edges 601a and 601b of the intraoral filter paper 601 are in contact with the upper portion 802a of the teeth tray 802 and the lower portion 802b of the teeth tray 802 respectively. Thus, the intraoral filter paper 601 in conjunction with the teeth tray 802 is used as an intraoral 3D facial mask 801 to filter air through the intraoral areas 702. A lever 803 that can be optionally used with the teeth tray 802 stabilizes the teeth tray 802 in the intraoral areas 702. The lever 803 can be used to adjustably position the teeth tray 802 in the intraoral areas 702.

The intraoral filter paper 601 is folded, for example, in a semicircular shape as exemplarily illustrated in FIGS. 6A-6B, and sealed with the teeth tray 802 as exemplarily illustrated in FIG. 8B. The intraoral filter paper 601 is sealed with the teeth tray 802, for example, by a zip lock seal (not shown) for forming an airtight seal between the intraoral filter paper 601 and the teeth tray 802 to avoid air leakage. The airtight seal between the intraoral filter paper 601 and the teeth tray 802 functions as a barrier and separates the air inside the mouth 701, that is, behind the teeth 703, and the air outside the mouth 701, that is, outside the teeth 703. In an embodiment, a user can use the intraoral 3D facial mask 801 comprising the intraoral filter paper 601 sealed with the teeth tray 802, when the user cannot breathe through his/her nose and has to breathe through the mouth 701. The user can also use the intraoral 3D facial mask 801 comprising the intraoral filter paper 601 and/or the teeth tray 802 when 3D facial masks, for example, 401 exemplarily illustrated in FIG. 4A, that cover the user's face 901 are not allowed. The intraoral filter paper 601 and the teeth tray 802 can be changed after each use.

Consider an example where a user wants to develop a three-dimensional (3D) facial mask 801 for covering internal areas, for example, intraoral areas 702 of a facial part, for example, a mouth 701 of the user's face 901 exemplarily illustrated in FIG. 9. The user provides multiple two-dimensional (2D) images in multiple views comprising, for example, a profile view, an elevation view, etc., of the intraoral areas 702 and extraoral areas of the mouth 701 of the user's face 901 to the mask development system. The mask development system receives the 2D images from the user and obtains facial parameters comprising, for example, a lip profile, a dental profile, etc., from the received 2D images. The dental profile comprises, for example, dimensions of the teeth 703, gingiva, an alveolar ridge form, etc. The mask development system constructs a 3D facial structure 201 exemplarily illustrated in FIG. 2, of an actual size of the user's face 901 using one or more of the obtained facial parameters. The mask development system constructs a 3D mask structure (not shown) configured to fit the intraoral areas 702 of the mouth 701 of the constructed 3D facial structure 201. The constructed 3D mask structure comprises a 3D structure for the teeth tray 802 and a 3D structure for the filter element, for example, the intraoral filter paper 601. The mask development system constructs the 3D mask structure using the constructed 3D facial structure 201 as a reference, that is, by using the facial parameters comprising, for example, the dimensions of the teeth 703, a distance between the alveolar ridges when the mouth 208 of the constructed 3D facial structure 201 exemplarily illustrated in FIG. 2, is in an open state and a closed state, etc.

The mask development system configures the 3D structure for the teeth tray 802 by creating a seal around a periphery of each of the upper teeth and the lower teeth in the constructed 3D structure for the teeth tray 802 to preclude leakage of air between the gaps between the teeth 703 exemplarily illustrated in FIG. 7. The mask development system further configures the 3D structure for the teeth tray 802 by configuring one or more grooves (not shown) in the 3D structure for the teeth tray 802, for incorporating an accessory, for example, a lever 803 used to adjustably position the teeth tray 802. The mask development system further configures the constructed 3D structure for the intraoral filter paper 601 by configuring one or more spaces in the constructed 3D structure for the intraoral filter paper 601, for facilitating stretching and recoiling of the intraoral filter paper 601 when inserted in the intraoral areas 702 of the mouth 701. The configured spaces in the constructed 3D structure for the intraoral filter paper 601 enable facial movements, for example, movement of the user's jaw. The user provides his/her inputs for one or more design parameters comprising, for example, a blue color for the intraoral filter paper 601, a silicone material for the teeth tray 802, etc., based on the user's preferences to the mask development system to configure the design parameters for the constructed 3D mask structure. The mask development system configures the design parameters for the constructed 3D mask structure based on the user inputs and transmits the configured 3D mask structure to a 3D printing device for constructing the intraoral 3D facial mask 801 that the user can wear to ensure filtration of air flowing through the intraoral areas 702 of the user's mouth 701.

FIG. 9 exemplarily illustrates a side perspective view of a three-dimensional (3D) facial mask 903 constructed using the mask development system for external areas of a nose 902 of a user's face 901, in conjunction with the 3D facial mask 801 constructed for intraoral areas 702 of a mouth 701 of the user's face 901. The mask development system develops a 3D facial mask 903 for covering an extranasal area of the user's nose 902 as exemplarily illustrated in FIG. 9. The 3D facial mask 903 is positioned over the nose 902, while the 3D facial mask 801 comprising the intraoral filter paper 601 and the intraoral teeth tray 802 is positioned inside the user's mouth 701 and is not visible as exemplarily illustrated in FIG. 9. The 3D facial mask 903 is used for covering the nose 902 and filtering air through the nose 902 when the user breathes through the nose 902, while the 3D facial mask 801 comprising the intraoral filter paper 601 and the intraoral teeth tray 802 filters air through the intraoral areas 702 of the user's mouth 701 when the user breathes through the mouth 701. The intraoral filter paper 601 in the user's mouth 701 facilitates filtering of air passing through the intraoral areas 702 of the user's mouth 701, while the teeth tray 802 precludes leakage of air between the teeth 703 in the user's mouth 701 exemplarily illustrated in FIG. 7. In an embodiment, the intraoral filter paper 601 and the intraoral teeth tray 802 of the 3D facial mask 801 are used independent of the 3D facial mask 903, when the user is breathing only through his/her mouth 701. The intraoral filter paper 601 and the intraoral teeth tray 802 used for filtering air are not visible, that is, not seen when the user's mouth 701 is closed and hence can be used comfortably without being visible and shown to the public.

Consider an example where a user wants to develop a three-dimensional (3D) facial mask 903 for covering extranasal areas of the nose 902 of the user's face 901. In this example, consider that the user wants to use the constructed 3D facial mask 903 in conjunction with the 3D facial mask 801 that covers intraoral areas 702 of the user's mouth 701. The user provides multiple two-dimensional (2D) images in multiple views comprising, for example, a profile view, an elevation view, etc., of the user's face 901 to the mask development system. The mask development system receives the 2D images of the user's face 901 from the user and obtains facial parameters comprising, for example, a nasal tip projection ratio, a nasal width, a nasal height, a distance between the user's nose 902 and the user's mouth 701, etc. The mask development system constructs a 3D facial structure 201 exemplarily illustrated in FIG. 2, of an actual size of the user's face 901 using one or more of the obtained facial parameters. The mask development system constructs a 3D mask structure (not shown), configured to fit the extranasal areas of the nose 203 of the constructed 3D facial structure 201 exemplarily illustrated in FIG. 2. The mask development system configures the constructed 3D mask structure by creating a seal in the constructed 3D mask structure, around the extranasal areas of the constructed 3D facial structure 201. The mask development system further configures the constructed 3D mask structure by configuring spaces in the constructed 3D mask structure, around the extranasal areas of the constructed 3D facial structure 201 using the obtained facial parameters, that is, the distance between the nose 902 and the mouth 701, the nasal tip projection ratio, etc., for enabling ease of breathing and facial movements, for example, movement of the nose 902. The user provides his/her inputs for one or more design parameters comprising, for example, a violet color, a silicone material, etc., based on the user's preferences to the mask development system to configure the design parameters for the constructed 3D mask structure. The mask development system configures the design parameters for the constructed 3D mask structure based on the user inputs and transmits the configured 3D mask structure to a 3D printing device for constructing the 3D facial mask 903 that the user can use in conjunction with the 3D facial mask 801 comprising the intraoral filter paper 601 and the intraoral teeth tray 802 for covering intraoral areas 702 of the mouth 701. The user wears the constructed 3D facial mask 903 that covers the user's extranasal areas to ensure filtration of air flowing through the user's nostrils 902a. The user wears the 3D facial mask 801 that covers the intraoral areas 702 of the user's mouth 701 to ensure filtration of air flowing through the user's intraoral areas 702. Thus, the constructed 3D facial mask 903 that covers the extranasal areas of the user's nose 902 when used in conjunction with the 3D facial mask 801 that covers the intraoral areas 702 of the user's mouth 701 enables the user to breathe filtered air via his/her nose 902 and mouth 701.

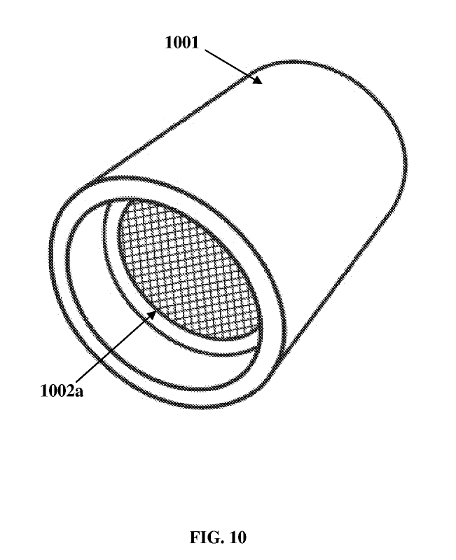

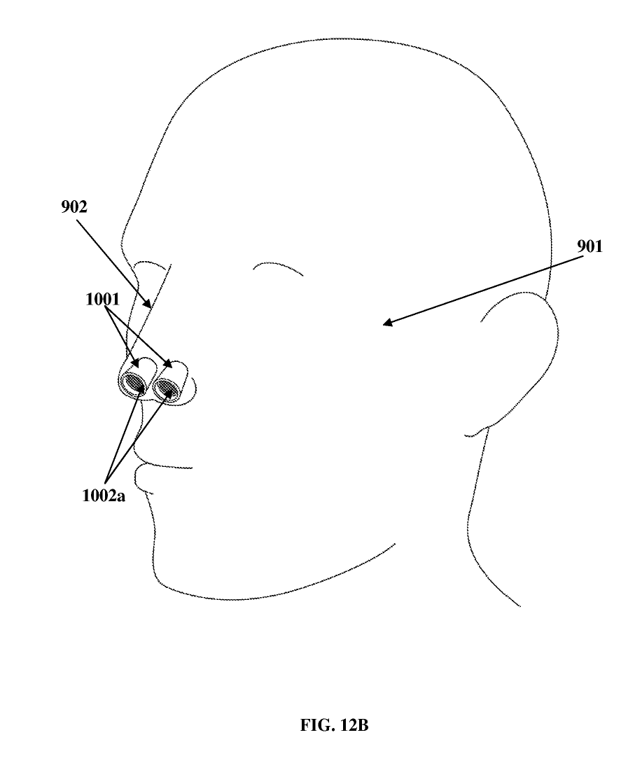

FIG. 10 exemplarily illustrates a perspective view of a three-dimensional (3D) facial mask 1001 constructed using the mask development system for intranasal areas, for example, nostrils 902a of a nose 902 of a user's face 901 exemplarily illustrated in FIGS. 12A-12B. The 3D facial mask 1001 is configured as an internal nose plug with a mini filter or an intranasal plug with a filter element, for example, a mesh element 1002a. The 3D facial mask 1001 is configured to be positioned inside the user's nostrils 902a to prevent nasal inhalation of allergens, pollutants, contaminants, and irritants such as dust, smoke, foul odors, etc. The mesh element 1002a, for example, a micro mesh filter filters air inhaled by a user wearing the 3D facial mask 1001.

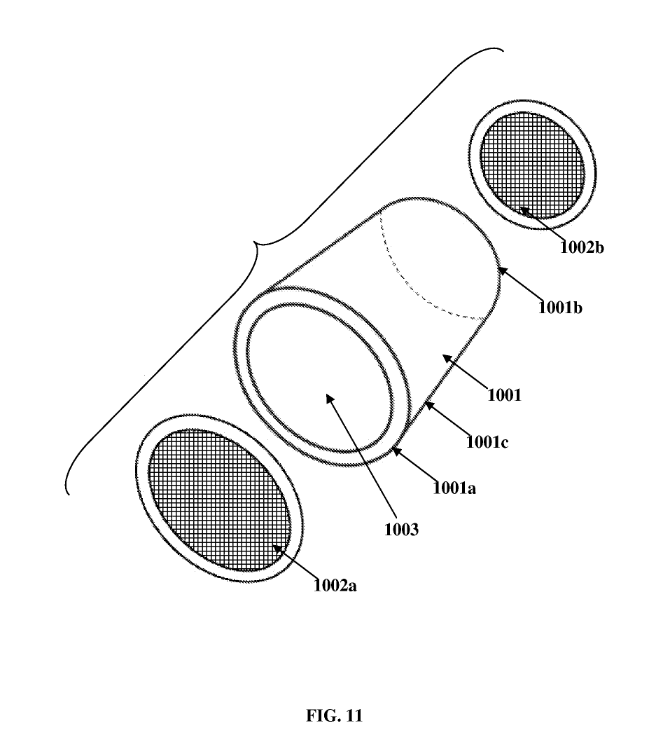

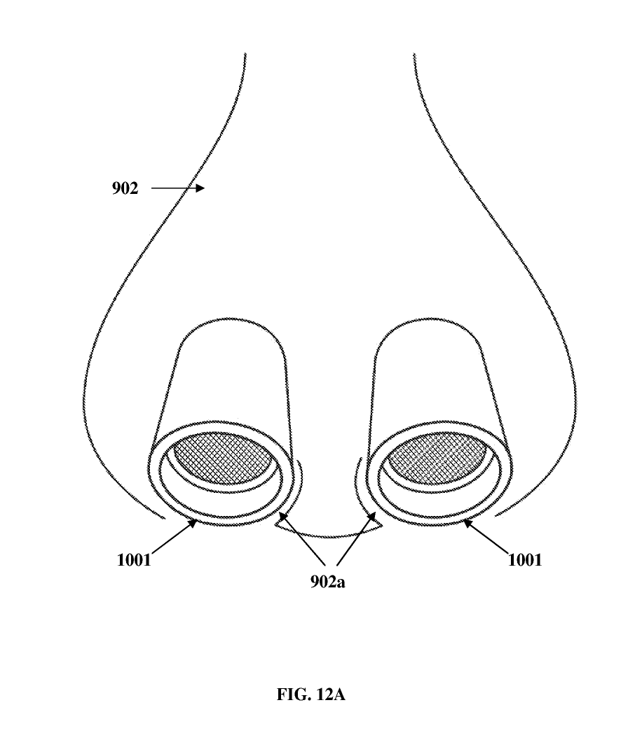

FIG. 11 exemplarily illustrates an exploded view of the three-dimensional (3D) facial mask 1001 constructed using the mask development system for intranasal areas of a nose 902 of a user's face 901 exemplarily illustrated in FIG. 12B. The 3D facial mask 1001 comprises mesh elements 1002a and 1002b positioned at a front end 1001a and a rear end 1001b of the 3D facial mask 1001 respectively. As exemplarily illustrated in FIG. 11, the 3D facial mask 1001 is of a generally cylindrical shape comprising a cylindrical wall 1001c extending from the front end 1001a of the 3D facial mask 1001 to the rear end 1001b of the 3D facial mask 1001. The cylindrical wall 1001c of the 3D facial mask 1001 is, for example, made of a foam based material. The cylindrical wall 1001c of the 3D facial mask 1001 defines a groove 1003 in the 3D facial mask 1001. The groove 1003 defined by the cylindrical wall 1001c of the 3D facial mask 1001 is a through hole that facilitates flow of air breathed through the mesh element 1002a at the front end 1001a of the 3D facial mask 1001 to the mesh element 1002b at the rear end 1001b of the 3D facial mask 1001, and into the windpipe of a user. The 3D facial mask 1001 is positioned in the intranasal areas, for example, the user's nostrils 902a exemplarily illustrated in FIG. 12A, such that the rear end 1001b of the 3D facial mask 1001 is not visible after insertion. The front end 1001a of the inserted 3D facial mask 1001 may be partially visible, completely visible, or not visible, after insertion in the user's nostrils 902a.

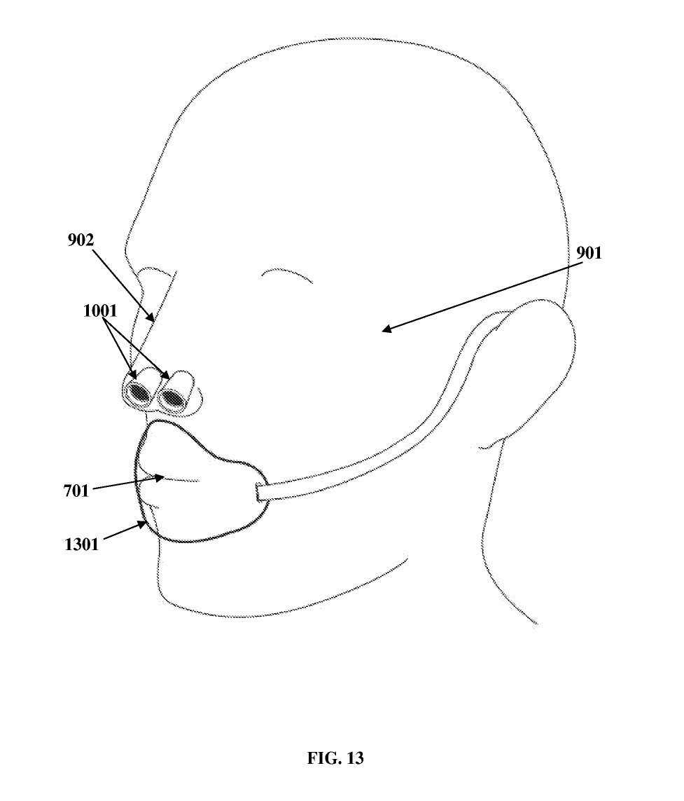

FIGS. 12A-12B exemplarily illustrate different views of the three-dimensional (3D) facial mask 1001 shown in FIG. 10, inserted into intranasal areas, for example, nostrils 902a of a nose 902 of a user's face 901 for filtering air passing through the nostrils 902a. Consider an example where a user wants to develop a 3D facial mask 1001 for covering intranasal areas, for example, nostrils 902a of the user's face 901. The user provides multiple two-dimensional (2D) images in multiple views comprising, for example, a profile view, an elevation view, etc., of the user's face 901 to the mask development system. The mask development system receives the 2D images of the user's face 901 from the user and obtains facial parameters comprising, for example, a nose ridge form, a width of the user's nose 902, a length of the nose 902, dimensions of the nostrils 902a of the nose 902, dimensions of the intranasal cavity, etc. The mask development system constructs a 3D facial structure 201 exemplarily illustrated in FIG. 2, of an actual size of the user's face 901 using one or more of the obtained facial parameters. The mask development system constructs a 3D mask structure (not shown) configured to fit inside the nostrils 902a of the nose 902 of the constructed 3D facial structure 201. The mask development system configures the constructed 3D mask structure by creating a seal in the constructed 3D mask structure, around the nostrils 203a of the nose 203 of the constructed 3D facial structure 201 exemplarily illustrated in FIG. 2. The mask development system further configures the constructed 3D mask structure by configuring a groove 1003 exemplarily illustrated in FIG. 11, inside the constructed 3D mask structure for accommodating the mesh elements 1002a and 1002b for enabling breathing of filtered air through the mesh elements 1002a and 1002b. The user provides his/her inputs for one or more design parameters comprising, for example, a white color, a rubber material, etc., based on the user's preferences to the mask development system to configure the design parameters for the constructed 3D mask structure. The mask development system configures the design parameters for the constructed 3D mask structure based on the user inputs and transmits the configured 3D mask structure to a 3D printing device for constructing the 3D facial mask 1001 configured as an intranasal filter. The user inserts the constructed 3D facial mask 1001 into the nostrils 902a of the user's nose 902 to ensure filtration of air breathed through the user's nose 902. As exemplarily illustrated in FIG. 12B, the 3D facial mask 1001 can be worn without the 3D facial mask 801 and the 3D facial mask 1301 exemplarily illustrated in FIG. 8B and FIG. 13 respectively. A user can wear the 3D facial mask 1001 for breathing filtered air, without wearing the 3D facial mask 801 or 1301, when the user breathes air only through his/her nose 902.

FIG. 13 exemplarily illustrates a three-dimensional (3D) facial mask 1301 constructed using the mask development system for external areas of a mouth 701 of a user's face 901, in conjunction with the 3D facial mask 1001 constructed for intranasal areas, for example, nostrils 902a exemplarily illustrated in FIG. 12A, of the nose 902 of the user's face 901. Consider an example where a user wants to develop a 3D facial mask 1301 for covering extraoral areas such as oral muscle areas of the user's face 901. In this example, consider that the user wants to use the constructed 3D facial mask 1301 in conjunction with the 3D facial mask 1001 constructed for the nostrils 902a of the nose 902 of the user's face 901 to ensure filtration of air breathed through the nose 902 and the mouth 701. The user provides multiple two-dimensional (2D) images in multiple views comprising, for example, a profile view, an elevation view, etc., of the user's face 901 to the mask development system. The mask development system receives the 2D images of the user's face 901 from the user and obtains facial parameters comprising, for example, a distance between the nose 902 and the mouth 701, a chin projection, a chin shape, a lip profile, a jaw profile, etc. The mask development system constructs a 3D facial structure 201 exemplarily illustrated in FIG. 2, of an actual size of the user's face 901 using one or more of the obtained facial parameters. The mask development system constructs a 3D mask structure (not shown) configured to fit the extraoral areas of the mouth 208 of the constructed 3D facial structure 201 exemplarily illustrated in FIG. 2.

The mask development system configures the constructed 3D mask structure by creating a seal in the constructed 3D mask structure, around the extraoral areas of the constructed 3D facial structure 201. The mask development system further configures the constructed 3D mask structure by configuring one or more spaces 206 and 207 exemplarily illustrated in FIG. 2, in the constructed 3D mask structure, around the extraoral areas, for example, the chin 204 and lips of the mouth 208 of the constructed 3D facial structure 201 using the obtained facial parameters, that is, the chin projection, the chin shape, the lip profile, etc., for enabling ease of breathing and facial movements, for example, oral muscle movements created while talking, smiling, yawning, etc., chin movements, jaw movements, etc. The user provides his/her inputs for one or more design parameters comprising, for example, a white color, a non-woven fabric material, etc., based on the user's preferences to the mask development system to configure the design parameters for the constructed 3D mask structure. The mask development system configures the design parameters for the constructed 3D mask structure based on the user inputs and transmits the configured 3D mask structure to a 3D printing device for constructing the 3D facial mask 1301 that the user can use in conjunction with the 3D facial mask 1001 constructed for the user's nostrils 902a exemplarily illustrated in FIG. 12A. The user wears the constructed 3D facial mask 1301 over the user's extraoral areas to ensure filtration of air flowing through the user's mouth 701. The user inserts the 3D facial mask 1001 into the nostrils 902a to ensure filtration of air flowing through the user's nose 902. Thus, the constructed 3D facial mask 1301 that covers the extraoral areas of the user's face 901, when used in conjunction with the 3D facial mask 1001 in the user's nostrils 902a, enables the user to breathe filtered air through his/her nose 902 and mouth 701.

Since air typically flows in an uncontrolled manner through both oral and nasal cavities of a user, a safe way to ensure that a user is breathing filtered air is to develop a 3D facial mask, for example, 301 and 401 exemplarily illustrated in FIG. 3-4A, that filters air flowing through the nose 902 and the mouth 701. The 3D facial mask 1001 constructed for the intranasal areas increases resistance to the air that the user breathes. Therefore, the 3D facial mask 1001 is optimally used with the 3D facial mask 1301 constructed for extraoral areas or the 3D facial mask 801 constructed for the intraoral areas 702 exemplarily illustrated in FIG. 9, to provide sufficient filtered air for breathing with minimum air resistance.

FIG. 14 exemplarily illustrates a front perspective view of an embodiment of the three-dimensional (3D) facial mask 1401 shown in FIG. 10, constructed using the mask development system for intranasal areas of a nose 902 of a user's face 901 exemplarily illustrated in FIG. 15B. In this embodiment, the 3D facial mask 1401 comprises an accessory 1402. The accessory 1402 is, for example, a clip 1402 made of one or more of multiple materials comprising, for example, plastic, metal, glass, etc. In this embodiment, the 3D facial mask 1401 constructed using the mask development system, comprises two of the 3D facial masks 1001 fixedly connected to each other via the accessory 1402. The 3D facial masks 1001 constituting the 3D facial mask 1401 comprise the mesh elements 1002a and 1002b exemplarily illustrated in FIG. 11.

FIGS. 15A-15B exemplarily illustrate different views of the three-dimensional (3D) facial mask 1401 shown in FIG. 14, inserted into the intranasal areas of a nose 902 of a user's face 901 for filtering air passing through the intranasal areas. As exemplarily illustrated in FIG. 15A, the 3D facial mask 1401 is inserted into the nostrils 902a of the nose 902 such that the accessory 1402 that connects the two 3D facial masks 1001 of the 3D facial mask 1401 snugly fits against the nasal columella 902b to facilitate a comfort and secure fit of the 3D facial mask 1401 in the nostrils 902a. As exemplarily illustrated in FIG. 15B, the 3D facial mask 1401 can be worn without the 3D facial mask 801 and the 3D facial mask 1301 exemplarily illustrated in FIG. 8B and FIG. 13 respectively. A user can wear the 3D facial mask 1401 for breathing filtered air, without wearing the 3D facial mask 801 or 1301, when the user breathes air only through his/her nose 902.

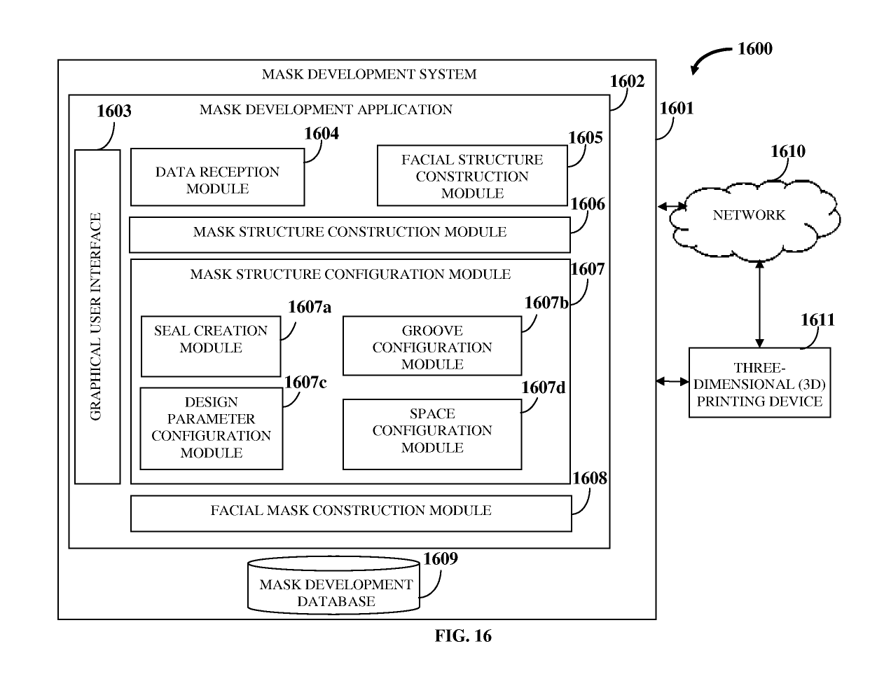

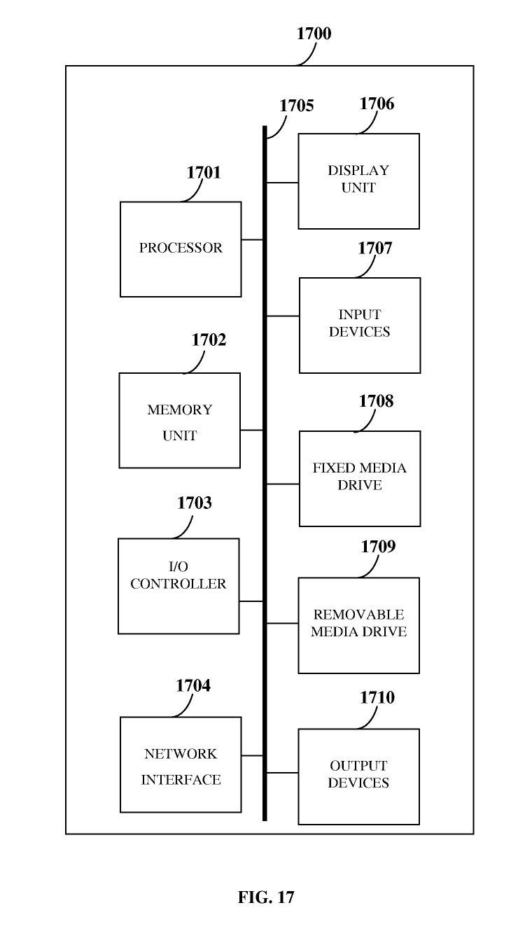

FIG. 16 exemplarily illustrates a computer implemented system 1600 for constructing a three-dimensional (3D) facial mask for air supply and air exchange. The computer implemented system 1600 disclosed herein comprises the mask development system 1601 in communication with a 3D printing device 1611 via a network 1610. In an embodiment, the mask development system 1601 communicates with the 3D printing device 1611 directly through a wired connection. The mask development system 1601 comprises a mask development application 1602, which is a software application that defines the computer program instructions for implementing the method disclosed herein. In an embodiment, the mask development application 1602 is downloadable on a user device (not shown), for example, a mask developer's device such as a personal computer, a tablet computing device, a mobile computer, a mobile phone, a smart phone, a portable computing device, a laptop, a personal digital assistant, a touch centric device, a workstation, a client device, a portable electronic device, a network enabled computing device, an interactive network enabled communication device, an image capture device, a video recorder, any other suitable computing equipment, and combinations of multiple pieces of computing equipment. The mask development system 1601 is accessible to users of the mask development system 1601, for example, through a broad spectrum of technologies and devices such as personal computers with access to the internet, internet enabled cellular phones, tablet computing devices, etc.

In an embodiment, the mask development system 1601 is configured as a web based platform, for example, a website hosted on a server or a network of servers. In another embodiment, the mask development system 1601 is configured to operate as a software as a service (SaaS). In another embodiment, the mask development system 1601 is configured to operate, for example, as a platform as a service (PaaS) implemented in a cloud computing environment. As used herein, “cloud computing environment” refers to a processing environment comprising configurable computing physical and logical resources, for example, networks, servers, storage, applications, services, etc., and data distributed over the network 1610. The cloud computing environment provides on-demand network access to a shared pool of the configurable computing physical and logical resources. In this embodiment, the mask development system 1601 is a cloud computing based platform implemented as a service for constructing a 3D facial mask for air supply and air exchange. The mask development system 1601 is constructed, for example, using the Google App engine cloud infrastructure of Google Inc., Amazon Web Services® of Amazon Technologies, Inc., the Amazon elastic compute cloud EC2® web service of Amazon Technologies, Inc., the Google® Cloud platform of Google Inc., the Microsoft® Cloud platform of Microsoft Corporation, etc.

The network 1610 through which the mask development system 1601 communicates with the 3D printing device 1611 is, for example, the internet, an intranet, a wired network, a wireless network, a communication network that implements Bluetooth® of Bluetooth Sig, Inc., a network that implements Wi-Fi® of Wi-Fi Alliance Corporation, an ultra-wideband communication network (UWB), a wireless universal serial bus (USB) communication network, a communication network that implements ZigBee® of ZigBee Alliance Corporation, a general packet radio service (GPRS) network, a mobile telecommunication network such as a global system for mobile (GSM) communications network, a code division multiple access (CDMA) network, a third generation (3G) mobile communication network, a fourth generation (4G) mobile communication network, a long-term evolution (LTE) mobile communication network, a public telephone network, etc., a local area network, a wide area network, an internet connection network, an infrared communication network, etc., or a network formed from any combination of these networks.

The mask development system 1601 disclosed herein comprises a non-transitory computer readable storage medium such as a memory unit, and at least one processor communicatively coupled to the non-transitory computer readable storage medium. As used herein, “non-transitory computer readable storage medium” refers to all computer readable media, for example, non-volatile media such as optical discs or magnetic disks, volatile media such as a register memory, a processor cache, etc., and transmission media such as wires that constitute a system bus coupled to the processor, except for a transitory, propagating signal. The non-transitory computer readable storage medium stores computer program instructions defined by modules, for example, 1604, 1605, 1606, 1607, 1607a, 1607b, 1607c, 1607d, 1608, etc., of the mask development system 1601. The processor is configured to execute the defined computer program instructions.

The mask development system 1601 comprises a graphical user interface (GUI) 1603, a data reception module 1604, a facial structure construction module 1605, a mask structure construction module 1606, a mask structure configuration module 1607, a facial mask construction module 1608, and a mask development database 1609. A mask developer inputs images of a user's face captured via a two-dimensional (2D) image capture device and/or a three-dimensional (3D) image capture device, and the user's biometric data, for example, height and facial dimensions of the user into the mask development system 1601 via the GUI 1603. The GUI 1603 is, for example, a webpage of a website hosted by the mask development system 1601, an online web interface, a web based downloadable application interface, a mobile based downloadable application interface, etc. The data reception module 1604 receives the images of the user's face, for example, multiple views of the user's face in one or more image formats and the user's biometric data via the GUI 1603.

The mask development database 1609 stores the received images and the biometric data of the user. The mask development database 1609 is any storage area or medium that can be used for storing data and files. The mask development database 1609 is, for example, a structured query language (SQL) data store or a not only SQL (NoSQL) data store such as the Microsoft® SQL Server®, the Oracle® servers, the MySQL® database of MySQL AB Company, the mongoDB® of MongoDB, Inc., the Neo4j graph database of Neo Technology Corporation, the Cassandra database of the Apache Software Foundation, the HBase™ database of the Apache Software Foundation, etc. In an embodiment, the mask development database 1609 can also be a location on a file system. In another embodiment, the mask development database 1609 can be remotely accessed by the mask development system 1601 via the network 1610. In another embodiment, the mask development database 1609 is configured as a cloud based database implemented in a cloud computing environment, where computing resources are delivered as a service over the network 1610.

The facial structure construction module 1605 constructs a three-dimensional (3D) facial structure of an actual size of the user's face using one or more of multiple facial parameters obtained from the received images and the biometric data. The mask structure construction module 1606 constructs a 3D mask structure configured to fit internal areas and/or external areas of one or more facial parts of the constructed 3D facial structure. The mask structure configuration module 1607 configures the constructed 3D mask structure. The mask structure configuration module 1607 comprises a seal creation module 1607a, a groove configuration module 1607b, and a design parameter configuration module 1607c. The seal creation module 1607a creates a seal in the constructed 3D mask structure, around the internal areas and/or the external areas of one or more facial parts of the constructed 3D facial structure. The groove configuration module 1607b configures one or more grooves in the constructed 3D mask structure, proximal to the internal areas and/or the external areas of one or more facial parts of the constructed 3D facial structure for incorporating one or more air supply and air exchange elements, for example, an in-tube, an out tube, a safety plug, a filter element, etc. In an embodiment, the mask structure configuration module 1607 further comprises a space configuration module 1607d. The space configuration module 1607d configures one or more spaces in the constructed 3D mask structure, around the internal areas and/or the external areas of one or more facial parts of the constructed 3D facial structure, using one or more facial parameters obtained from the received images and the biometric data for enabling ease of breathing and facial movements. The design parameter configuration module 1607c configures one or more design parameters for the constructed 3D mask structure, for example, based on the user's gender, the user's age, and one or more user inputs. The facial mask construction module 1608 transmits the configured 3D mask structure to the 3D printing device 1611 directly through a wired connection or via the network 1610 for constructing the 3D facial mask for air supply and air exchange. The 3D printing device 1611 prints a prototype of the 3D facial mask using 3D printing technology.

FIG. 17 exemplarily illustrates the hardware architecture 1700 of the mask development system 1601 exemplarily illustrated in FIG. 16, employed for constructing a three-dimensional (3D) facial mask for air supply and air exchange. The mask development system 1601 is a computer system programmable using a high level computer programming language. The mask development system 1601 may be implemented using programmed and purposeful hardware. In an embodiment, the mask development system 1601 is accessible on user devices of users, for example, mask developers, mask designers, mask customizers, etc., via a network 1610 exemplarily illustrated in FIG. 16, for example, a short range network or a long range network.

As exemplarily illustrated in FIG. 17, the hardware architecture 1700 of the mask development system 1601 comprises a processor 1701, a non-transitory computer readable storage medium such as a memory unit 1702 for storing programs and data, an input/output (I/O) controller 1703, a network interface 1704, a data bus 1705, a display unit 1706, input devices 1707, a fixed media drive 1708 such as a hard drive, a removable media drive 1709 for receiving removable media, output devices 1710, etc. The processor 1701 refers to any one or more microprocessors, central processing unit (CPU) devices, finite state machines, computers, microcontrollers, digital signal processors, logic, a logic device, an electronic circuit, an application specific integrated circuit (ASIC), a field-programmable gate array (FPGA), a chip, etc., or any combination thereof, capable of executing computer programs or a series of commands, instructions, or state transitions. The processor 1701 may also be implemented as a processor set comprising, for example, a general purpose microprocessor and a math or graphics co-processor. The processor 1701 is selected, for example, from the Intel® processors such as the Itanium® microprocessor or the Pentium® processors, Advanced Micro Devices (AMD®) processors such as the Athlon® processor, UltraSPARC® processors, microSPARC® processors, Hp® processors, International Business Machines (IBM®) processors such as the PowerPC® microprocessor, the MIPS® reduced instruction set computer (RISC) processor of MIPS Technologies, Inc., RISC based computer processors of ARM Holdings, Motorola® processors, Qualcomm® processors, etc. The mask development system 1601 disclosed herein is not limited employing the processor 1701. The mask development system 1601 may also employ a controller or a microcontroller. The processor 1701 executes the modules, for example, 1604, 1605, 1606, 1607, 1607a, 1607b, 1607c, 1607d, 1608, etc., of the mask development system 1601.

The memory unit 1702 is used for storing programs, applications, and data. For example, the data reception module 1604, the facial structure construction module 1605, the mask structure construction module 1606, the seal creation module 1607a, the groove configuration module 1607b, the design parameter configuration module 1607c, the space configuration module 1607d, the facial mask construction module 1608, etc., of the mask development system 1601 are stored in the memory unit 1702 of the mask development system 1601. The memory unit 1702 is, for example, a random access memory (RAM) or another type of dynamic storage device that stores information and instructions for execution by the processor 1701. The memory unit 1702 also stores temporary variables and other intermediate information used during execution of the instructions by the processor 1701. The mask development system 1601 further comprises a read only memory (ROM) or another type of static storage device that stores static information and instructions for the processor 1701. The I/O controller 1703 controls input actions and output actions performed by the mask development system 1601.

The network interface 1704 enables connection of the mask development system 1601 to the network 1610. In an embodiment, the network interface 1704 is provided as an interface card also referred to as a line card. The network interface 1704 comprises, for example, one or more of an infrared (IR) interface, an interface implementing Wi-Fi® of Wi-Fi Alliance Corporation, a universal serial bus (USB) interface, a FireWire® interface of Apple, Inc., an Ethernet interface, a frame relay interface, a cable interface, a digital subscriber line (DSL) interface, a token ring interface, a peripheral controller interconnect (PCI) interface, a local area network (LAN) interface, a wide area network (WAN) interface, interfaces using serial protocols, interfaces using parallel protocols, and Ethernet communication interfaces, asynchronous transfer mode (ATM) interfaces, a high speed serial interface (HSSI), a fiber distributed data interface (FDDI), interfaces based on transmission control protocol (TCP)/internet protocol (IP), interfaces based on wireless communications technology such as satellite technology, radio frequency (RF) technology, near field communication, etc. The data bus 1705 permits communications between the modules, for example, 1603, 1604, 1605, 1606, 1607, 1607a, 1607b, 1607c, 1607d, 1608, 1609, etc., of the mask development system 1601.

The display unit 1706, via the graphical user interface (GUI) 1603, displays information, display interfaces, user interface elements such as text fields, checkboxes, text boxes, windows, etc., for allowing the users to input images of the users' face, view and enter design parameters comprising, for example, physical dimensions of a face, color, size, shape, design patterns, etc., for the 3D facial mask. The display unit 1706 comprises, for example, a liquid crystal display, a plasma display, an organic light emitting diode (OLED) based display, etc. The input devices 1707 are used for inputting data into the mask development system 1601. The users use the input devices 1707 to provide inputs to the mask development system 1601. For example, a user may upload images of the user's face, biometric data, design parameters, etc., for the 3D facial mask using the input devices 1707. The input devices 1707 are, for example, a keyboard such as an alphanumeric keyboard, a microphone, a joystick, a pointing device such as a computer mouse, a touch pad, a light pen, a physical button, a touch sensitive display device, a track ball, a pointing stick, any device capable of sensing a tactile input, etc.

Computer applications and programs are used for operating the mask development system 1601. The programs are loaded onto the fixed media drive 1708 and into the memory unit 1702 via the removable media drive 1709. In an embodiment, the computer applications and programs may be loaded directly via the network 1610. Computer applications and programs are executed by double clicking a related icon displayed on the display unit 1706 using one of the input devices 1707. The output devices 1710 output the results of operations performed by the mask development system 1601. For example, the mask development system 1601 displays one or more views of the constructed 3D facial mask to the users using the output devices 1710.

The processor 1701 executes an operating system, for example, the Linux® operating system, the Unix® operating system, any version of the Microsoft® Windows® operating system, the Mac OS of Apple Inc., the IBM® OS/2, VxWorks® of Wind River Systems, Inc., QNX Neutrino® developed by QNX Software Systems Ltd., the Palm OS®, the Solaris operating system developed by Sun Microsystems, Inc., the Android operating system, the Windows Phone® operating system of Microsoft Corporation, the BlackBerry® operating system of BlackBerry Limited, the iOS operating system of Apple Inc., the Symbian™ operating system of Symbian Foundation Limited, etc. The mask development system 1601 employs the operating system for performing multiple tasks. The operating system is responsible for management and coordination of activities and sharing of resources of the mask development system 1601. The operating system further manages security of the mask development system 1601, peripheral devices connected to the mask development system 1601, and network connections. The operating system employed on the mask development system 1601 recognizes, for example, inputs provided by the users using one of the input devices 1707, the output display, files, and directories stored locally on the fixed media drive 1708. The operating system on the mask development system 1601 executes different programs using the processor 1701. The processor 1701 and the operating system together define a computer system for which application programs in high level programming languages are written.

The processor 1701 of the mask development system 1601 retrieves instructions defined by the data reception module 1604, the facial structure construction module 1605, the mask structure construction module 1606, the seal creation module 1607a, the groove configuration module 1607b, the design parameter configuration module 1607c, the space configuration module 1607d, the facial mask construction module 1608, etc., for performing respective functions disclosed in the detailed description of FIG. 16. The processor 1701 retrieves instructions for executing the modules, for example, 1604, 1605, 1606, 1607, 1607a, 1607b, 1607c, 1607d, 1608, etc., of the mask development system 1601 from the memory unit 1702. A program counter determines the location of the instructions in the memory unit 1702. The program counter stores a number that identifies the current position in the program of each of the modules, for example, 1604, 1605, 1606, 1607, 1607a, 1607b, 1607c, 1607d, 1608, etc., of the mask development system 1601. The instructions fetched by the processor 1701 from the memory unit 1702 after being processed are decoded. The instructions are stored in an instruction register in the processor 1701. After processing and decoding, the processor 1701 executes the instructions, thereby performing one or more processes defined by those instructions.

At the time of execution, the instructions stored in the instruction register are examined to determine the operations to be performed. The processor 1701 then performs the specified operations. The operations comprise arithmetic operations and logic operations. The operating system performs multiple routines for performing a number of tasks required to assign the input devices 1707, the output devices 1710, and memory for execution of the modules, for example, 1604, 1605, 1606, 1607, 1607a, 1607b, 1607c, 1607d, 1608, etc., of the mask development system 1601. The tasks performed by the operating system comprise, for example, assigning memory to the modules, for example, 1604, 1605, 1606, 1607, 1607a, 1607b, 1607c, 1607d, 1608, etc., of the mask development system 1601, and to data used by the mask development system 1601, moving data between the memory unit 1702 and disk units, and handling input/output operations. The operating system performs the tasks on request by the operations and after performing the tasks, the operating system transfers the execution control back to the processor 1701. The processor 1701 continues the execution to obtain one or more outputs. The outputs of the execution of the modules, for example, 1604, 1605, 1606, 1607, 1607a, 1607b, 1607c, 1607d, 1608, etc., of the mask development system 1601 are displayed to the users on the display unit 1706.

For purposes of illustration, the detailed description refers to the mask development system 1601 being run locally as a single computer system; however the scope of the computer implemented method and system 1600 disclosed herein is not limited to the mask development system 1601 being run locally as a single computer system via the operating system and the processor 1701, but may be extended to run remotely over the network 1610 by employing a web browser and a remote server, a mobile phone, or other electronic devices. One or more portions of the mask development system 1601 may be distributed across one or more computer systems (not shown) coupled to the network 1610.

Disclosed herein is also a computer program product comprising a non-transitory computer readable storage medium that stores computer program codes comprising instructions executable by at least one processor 1701 for constructing a three-dimensional (3D) facial mask for air supply and air exchange. The computer program product comprises a first computer program code for receiving multiple images of a user's face and the user's biometric data; a second computer program code for constructing a 3D facial structure of an actual size of the user's face using one or more of multiple facial parameters obtained from the received images and the biometric data; a third computer program code for constructing a 3D mask structure configured to fit internal areas and/or external areas of one or more facial parts of the constructed 3D facial structure; and a fourth computer program code for configuring the constructed 3D mask structure. The fourth computer program code comprises a fifth computer program code for creating a seal in the constructed 3D mask structure, around the internal areas and/or the external areas of one or more facial parts of the constructed 3D facial structure; a sixth computer program code for configuring one or more grooves in the constructed 3D facial structure, proximal to the internal areas and/or the external areas of one or more facial parts of the constructed 3D facial structure, for incorporating one or more air supply and air exchange elements; a seventh computer program code for configuring one or more design parameters for the constructed 3D mask structure; and an eighth program code for transmitting the configured 3D mask structure to the 3D printing device 1611 exemplarily illustrated in FIG. 16, for constructing a 3D facial mask for air supply and air exchange. In an embodiment, the fourth computer program code further comprises a ninth computer program code for configuring one or more spaces in the constructed three-dimensional mask structure, around the internal areas and/or the external areas of one or more facial parts of the constructed 3D facial structure using one or more of the facial parameters for enabling ease of breathing and facial movements.

The computer program product disclosed herein further comprises one or more additional computer program codes for performing additional steps that may be required and contemplated for constructing the 3D facial mask for air supply and air exchange. In an embodiment, a single piece of computer program code comprising computer executable instructions performs one or more steps of the computer implemented method disclosed herein for constructing the 3D facial mask for air supply and air exchange. The computer program codes comprising computer executable instructions are embodied on the non-transitory computer readable storage medium. The processor 1701 of the mask development system 1601 retrieves these computer executable instructions and executes them. When the computer executable instructions are executed by the processor 1701, the computer executable instructions cause the processor 1701 to perform the steps of the computer implemented method for constructing the 3D facial mask for air supply and air exchange.

It will be readily apparent that the various methods, algorithms, and computer programs disclosed herein may be implemented on computer readable media appropriately programmed for computing devices. As used herein, “computer readable media” refers to non-transitory computer readable media that participate in providing data, for example, instructions that may be read by a computer, a processor 1701 or a similar device. Non-transitory computer readable media comprise all computer readable media, for example, non-volatile media, volatile media, and transmission media, except for a transitory, propagating signal. Non-volatile media comprise, for example, optical discs or magnetic disks and other persistent memory volatile media including a dynamic random access memory (DRAM), which typically constitutes a main memory. Volatile media comprise, for example, a register memory, a processor cache, a random access memory (RAM), etc. Transmission media comprise, for example, coaxial cables, copper wire, fiber optic cables, modems, etc., including wires that constitute a system bus coupled to the processor 1701, etc. Common forms of computer readable media comprise, for example, a floppy disk, a flexible disk, a hard disk, magnetic tape, a laser disc, a Blu-ray Disc® of the Blu-ray Disc Association, any magnetic medium, a compact disc-read only memory (CD-ROM), a digital versatile disc (DVD), any optical medium, a flash memory card, punch cards, paper tape, any other physical medium with patterns of holes, a random access memory (RAM), a programmable read only memory (PROM), an erasable programmable read only memory (EPROM), an electrically erasable programmable read only memory (EEPROM), a flash memory, any other memory chip or cartridge, or any other medium from which a computer can read.

The computer programs that implement the methods and algorithms disclosed herein may be stored and transmitted using a variety of media, for example, the computer readable media in a number of manners. In an embodiment, hard-wired circuitry or custom hardware may be used in place of, or in combination with, software instructions for implementation of the processes of various embodiments. Therefore, the embodiments are not limited to any specific combination of hardware and software. In general, the computer program codes comprising computer executable instructions may be implemented in any programming language. Examples of programming languages that can be used comprise C, C++, C#, Java®, JavaScript®, Fortran, Ruby, Perl®, Python®, Visual Basic®, hypertext preprocessor (PHP), Microsoft®.NET etc. Other object-oriented, functional, scripting, and/or logical programming languages may also be used. The computer program codes or software programs may be stored on or in one or more mediums as object code. Various aspects of the method and the mask development system 1601 disclosed herein may be implemented in a non-programmed environment comprising documents created, for example, in a hypertext markup language (HTML), an extensible markup language (XML), or other format that render aspects of the graphical user interface (GUI) 1603 or perform other functions, when viewed in a visual area or a window of a browser program. Various aspects of the method and the mask development system 1601 disclosed herein may be implemented as programmed elements, or non-programmed elements, or any suitable combination thereof. The computer program product disclosed herein comprises one or more computer program codes for implementing the processes of various embodiments.

Where databases are described such as the mask development database 1609, it will be understood by one of ordinary skill in the art that (i) alternative database structures to those described may be readily employed, and (ii) other memory structures besides databases may be readily employed. Any illustrations or descriptions of any sample databases disclosed herein are illustrative arrangements for stored representations of information. Any number of other arrangements may be employed besides those suggested by tables illustrated in the drawings or elsewhere. Similarly, any illustrated entries of the databases represent exemplary information only; one of ordinary skill in the art will understand that the number and content of the entries can be different from those disclosed herein. Further, despite any depiction of the databases as tables, other formats including relational databases, object-based models, and/or distributed databases may be used to store and manipulate the data types disclosed herein. Likewise, object methods or behaviors of a database can be used to implement various processes such as those disclosed herein. In addition, the databases may, in a known manner, be stored locally or remotely from a device that accesses data in such a database. In embodiments where there are multiple databases in the mask development system 1601, the databases may be integrated to communicate with each other for enabling simultaneous updates of data linked across the databases, when there are any updates to the data in one of the databases.

The method and the system 1600 disclosed herein can be configured to work in a network environment comprising one or more computers that are in communication with one or more devices via the network 1610. The computers may communicate with the devices directly or indirectly, via a wired medium or a wireless medium such as the Internet, a local area network (LAN), a wide area network (WAN) or the Ethernet, a token ring, or via any appropriate communications mediums or combination of communications mediums. Each of the devices comprises processors, examples of which are disclosed above, that are adapted to communicate with the computers. In an embodiment, each of the computers is equipped with a network communication device, for example, a network interface card, a modem, or other network connection device suitable for connecting to the network 1610. Each of the computers and the devices executes an operating system, examples of which are disclosed above. While the operating system may differ depending on the type of computer, the operating system provides the appropriate communications protocols to establish communication links with the network 1610. Any number and type of machines may be in communication with the computers.

The method and the mask development system 1601 disclosed herein are not limited to a particular computer system platform, processor 1701, operating system, or network 1610. One or more aspects of the method and the mask development system 1601 disclosed herein may be distributed among one or more computer systems, for example, servers configured to provide one or more services to one or more client computers, or to perform a complete task in a distributed system. For example, one or more aspects of the method and the mask development system 1601 disclosed herein may be performed on a client-server system that comprises components distributed among one or more server systems that perform multiple functions according to various embodiments. These components comprise, for example, executable, intermediate, or interpreted code, which communicate over the network 1610 using a communication protocol. The method and the mask development system 1601 disclosed herein are not limited to be executable on any particular system or group of systems, and is not limited to any particular distributed architecture, network, or communication protocol.

The foregoing examples have been provided merely for the purpose of explanation and are in no way to be construed as limiting of the method and the system 1600 disclosed herein. While the method and the system 1600 have been described with reference to various embodiments, it is understood that the words, which have been used herein, are words of description and illustration, rather than words of limitation. Further, although the method and the system 1600 have been described herein with reference to particular means, materials, and embodiments, the method and the system 1600 are not intended to be limited to the particulars disclosed herein; rather, the method and the system 1600 extend to all functionally equivalent structures, methods and uses, such as are within the scope of the appended claims. Those skilled in the art, having the benefit of the teachings of this specification, may effect numerous modifications thereto and changes may be made without departing from the scope and spirit of the method and the system 1600 disclosed herein in their aspects.

We claim:

1. A computer implemented method for constructing a three-dimensional facial mask for air supply and air exchange, said method employing a computer implemented mask development system comprising at least one processor configured to execute computer program instructions for performing said method, said method comprising:
receiving a plurality of images of a face and biometric data by said mask development system;
constructing a three-dimensional facial structure of an actual size of said face by said mask development system using one or more of a plurality of facial parameters obtained from said received images and said biometric data;
constructing a three-dimensional mask structure configured to fit one or more of internal areas and external areas of one or more facial parts of said constructed three-dimensional facial structure by said mask development system;
configuring said constructed three-dimensional mask structure by said mask development system by:
creating a seal in said constructed three-dimensional mask structure, around said one or more of said internal areas and said external areas of said one or more facial parts of said constructed three-dimensional facial structure;
configuring one or more grooves in said constructed three-dimensional mask structure, proximal to said one or more of said internal areas and said external areas of said one or more facial parts of said constructed three-dimensional facial structure for incorporating one or more of a plurality of air supply and air exchange elements; and
configuring one or more of a plurality of design parameters for said constructed three-dimensional mask structure; and
transmitting said configured three-dimensional mask structure to a three-dimensional printing device by said mask development system for constructing said three-dimensional facial mask for said air supply and said air exchange.

2. The computer implemented method of claim 1, wherein said biometric data comprises a height of a user and facial dimensions of said user.

3. The computer implemented method of claim 1, wherein said facial parameters comprise a nasal tip projection, a cheekbone projection, an angle between a forehead and a nose, a distance between said nose and a mouth, a chin projection, a nose ridge form, a width of said nose, a length of said nose, dimensions of nostrils of said nose, a distance between said nostrils, a chin shape, a lip profile, and a dental profile comprising dimensions of teeth and an alveolar ridge form.

4. The computer implemented method of claim 1, wherein said configuration of said constructed three-dimensional mask structure by said mask development system further comprises configuring one or more spaces in said constructed three-dimensional mask structure, around said one or more of said internal areas and said external areas of said one or more facial parts of said constructed three-dimensional facial structure using said one or more of said facial parameters for enabling ease of breathing and facial movements.

5. The computer implemented method of claim 1, wherein said one or more of said design parameters are configured based on one or more of gender of a user, age of said user, and one or more user inputs.

6. The computer implemented method of claim 1, wherein said design parameters comprise facial characteristics, physical dimensions, color, size, shape, one or more design patterns, and one or more accessories.

7. The computer implemented method of claim 1, wherein said images of said face comprise a plurality of views of said face.

8. The computer implemented method of claim 1, wherein said one or more facial parts of said constructed three-dimensional facial structure correspond to one or more of a nose and a mouth of said face.

9. The computer implemented method of claim 1, wherein said internal areas comprise intranasal areas and intraoral areas, and wherein said external areas comprise extranasal areas and extraoral areas.

10. The computer implemented method of claim 1, wherein said air supply and air exchange elements comprise an in-tube, an out tube, a safety plug, and a filter element.

11. A computer implemented method for constructing a three-dimensional facial mask for air supply and air exchange, said method employing a mask development system comprising at least one processor configured to execute computer program instructions for performing said method, said method comprising:
receiving a plurality of images of a face and biometric data by said mask development system;
constructing a three-dimensional facial structure of an actual size of said face by said mask development system using one or more of a plurality of facial parameters obtained from said received images and said biometric data;
constructing a three-dimensional mask structure configured to fit one or more external areas of one or more facial parts of said constructed three-dimensional facial structure by said mask development system;
configuring said constructed three-dimensional mask structure by said mask development system by:
creating a seal in said constructed three-dimensional mask structure, around said one or more external areas of said one or more facial parts of said constructed three-dimensional facial structure;
configuring one or more grooves in said constructed three-dimensional mask structure, proximal to said one or more external areas of said one or more facial parts of said constructed three-dimensional facial structure for incorporating one or more of a plurality of air supply and air exchange elements;
configuring one or more spaces in said constructed three-dimensional mask structure, around said one or more external areas of said one or more facial parts of said constructed three-dimensional facial structure using said one or more of said facial parameters for enabling ease of breathing and facial movements; and
configuring one or more of a plurality of design parameters for said constructed three-dimensional mask structure; and
transmitting said configured three-dimensional mask structure to a three-dimensional printing device by said mask development system for constructing said three-dimensional facial mask for said air supply and said air exchange.

12. The computer implemented method of claim 11, wherein said biometric data comprises a height of a user and facial dimensions of said user.

13. The computer implemented method of claim 11, wherein said facial parameters comprise a nasal tip projection, a cheekbone projection, an angle between a forehead and a nose, a distance between said nose and a mouth, a chin projection, a nose ridge form, a chin shape, and a lip profile.

14. The computer implemented method of claim 11, wherein said one or more of said design parameters are configured based on one or more of gender of a user, age of said user, and one or more user inputs.

15. The computer implemented method of claim 11, wherein said design parameters comprise facial characteristics, physical dimensions, color, size, shape, one or more design patterns, and one or more accessories.

16. The computer implemented method of claim 11, wherein said images of said face comprise a plurality of views of said face.

17. The computer implemented method of claim 11, wherein said one or more facial parts of said constructed three-dimensional facial structure corresponding to one or more of a nose and a mouth of said face.

18. The computer implemented method of claim 11, wherein said one or more external areas comprise one or more of extranasal areas and extraoral areas.

19. The computer implemented method of claim 11, wherein said air supply and air exchange elements comprise an in-tube, an out tube, and a safety plug.

20. A computer implemented mask development system for constructing a three-dimensional facial mask for air supply and air exchange, said computer implemented mask development system comprising:
a non-transitory computer readable storage medium configured to store computer program instructions defined by modules of said computer implemented mask development system;
at least one processor communicatively coupled to said non-transitory computer readable storage medium, said at least one processor configured to execute said modules of said computer implemented mask development system;
said modules of said computer implemented mask development system comprising:
a data reception module configured to receive a plurality of images of a face and biometric data;
a facial structure construction module configured to construct a three-dimensional facial structure of an actual size of said face using one or more of a plurality of facial parameters obtained from said received images and said biometric data;
a mask structure construction module configured to construct a three-dimensional mask structure configured to fit one or more of internal areas and external areas of one or more facial parts of said constructed three-dimensional facial structure;
a mask structure configuration module configured to configure said constructed three-dimensional mask structure, said mask structure configuration module comprising:
a seal creation module configured to create a seal in said constructed three-dimensional mask structure, around said one or more of said internal areas and said external areas of said one or more facial parts of said constructed three-dimensional facial structure;
a groove configuration module configured to configure one or more grooves in said constructed three-dimensional mask structure, proximal to said one or more of said internal areas and said external areas of said one or more facial parts of said constructed three-dimensional facial structure for incorporating one or more of a plurality of air supply and air exchange elements; and
a design parameter configuration module configured to configure one or more of a plurality of design parameters for said constructed three-dimensional mask structure; and
a facial mask construction module configured to transmit said configured three-dimensional mask structure to a three-dimensional printing device for constructing said three-dimensional facial mask for said air supply and said air exchange.

21. The computer implemented mask development system of claim 20, wherein said biometric data comprises a height of a user and facial dimensions of said user.

22. The computer implemented mask development system of claim 20, wherein said facial parameters comprise a nasal tip projection, a cheekbone projection, an angle between a forehead and a nose, a distance between said nose and a mouth, a chin projection, a nose ridge form, a width of said nose, a length of said nose, dimensions of nostrils of said nose, a distance between said nostrils, a chin shape, a lip profile, and a dental profile comprising dimensions of teeth and an alveolar ridge form.

23. The computer implemented mask development system of claim 20, wherein said mask structure configuration module further comprises a space configuration module configured to configure one or more spaces in said constructed three-dimensional mask structure, around said one or more of said internal areas and said external areas of said one or more facial parts of said constructed three-dimensional facial structure using said one or more of said facial parameters for enabling ease of breathing and facial movements.

24. The computer implemented mask development system of claim 20, wherein said design parameter configuration module of said mask structure configuration module configures said one or more of said design parameters based on one or more of gender of a user, age of said user, and one or more user inputs.

25. The computer implemented mask development system of claim 20, wherein said design parameters comprise facial characteristics, physical dimensions, color, size, shape, one or more design patterns, and one or more accessories.

26. The computer implemented mask development system of claim 20, wherein said images of said face comprise a plurality views of said face.

27. The computer implemented mask development system of claim 20, wherein said one or more facial parts of said constructed three-dimensional facial structure correspond to one or more of a nose and a mouth of said face.

28. The computer implemented mask development system of claim 20, wherein said internal areas comprise intranasal areas and intraoral areas, and wherein said external areas comprise extranasal areas and extraoral areas.

29. The computer implemented mask development system of claim 20, wherein said air supply and air exchange elements comprise an in-tube, an out tube, a safety plug, and a filter element.

30. A computer program product comprising a non-transitory computer readable storage medium, said non-transitory computer readable storage medium storing computer program codes that comprise instructions executable by at least one processor, said computer program codes comprising:
a first computer program code for receiving a plurality of images of a face and biometric data, wherein said images comprise a plurality views of said face, and wherein said biometric data comprises a height of a user and facial dimensions of said user;
a second computer program code for constructing a three-dimensional facial structure of an actual size of said face using one or more of a plurality of facial parameters obtained from said received images and said biometric data, wherein said facial parameters comprise a nasal tip projection, a cheekbone projection, an angle between a forehead and a nose, a distance between said nose and a mouth, a chin projection, a nose ridge form, a width of said nose, a length of said nose, dimensions of nostrils of said nose, a distance between said nostrils, a chin shape, a lip profile, and a dental profile comprising dimensions of teeth and an alveolar ridge form;
a third computer program code for constructing a three-dimensional mask structure configured to fit one or more of internal areas and external areas of one or more facial parts of said constructed three-dimensional facial structure, wherein said one or more facial parts of said constructed three-dimensional facial structure correspond to one or more of said nose and said mouth of said face, and wherein said internal areas comprise intranasal areas and intraoral areas, and wherein said external areas comprise extranasal areas and extraoral areas;
a fourth computer program code for configuring said constructed three-dimensional mask structure, wherein said fourth computer program code comprises:
a fifth computer program code for creating a seal in said constructed three-dimensional mask structure, around said one or more of said internal areas and said external areas of said one or more facial parts of said constructed three-dimensional facial structure;
a sixth computer program code for configuring one or more grooves in said constructed three-dimensional mask structure, proximal to said one or more of said internal areas and said external areas of said one or more facial parts of said constructed three-dimensional facial structure for incorporating one or more of a plurality of air supply and air exchange elements; and
a seventh computer program code for configuring one or more of a plurality of design parameters for said constructed three-dimensional mask structure, wherein said one or more of said design parameters are configured based on one or more of gender of said user, age of said user, and one or more user inputs, and wherein said design parameters comprise facial characteristics, physical dimensions, color, size, shape, one or more design patterns, and one or more accessories; and
an eighth program code for transmitting said configured three-dimensional mask structure to a three-dimensional printing device for constructing a three-dimensional facial mask for air supply and air exchange.

31. The computer program product of claim 30, wherein said fourth computer program code further comprises a ninth computer program code for configuring one or more spaces in said constructed three-dimensional mask structure, around said one or more of said internal areas and said external areas of said one or more facial parts of said constructed three-dimensional facial structure using said one or more of said facial parameters for enabling ease of breathing and facial movements.
